(19) United States
(12) Patent Application Publication
(10) Pub. No.: US 2024/0081689 A1
(43) Pub. Date: Mar. 14, 2024

(54) METHOD AND SYSTEM FOR RESPIRATION AND MOVEMENT

(71) Applicant: LULULEMON ATHLETICA CANADA INC., Vancouver (CA)

(72) Inventors: Navjot KAILAY, Vancouver (CA); Joseph John SANTRY, Vancouver (CA); Samuel Haskel BERGMANN-GOOD, Vancouver (CA); William LY, Vancouver (CA); Anouk Johanna DE BROUWER, Vancouver (CA)

(21) Appl. No.: 18/462,893

(22) Filed: Sep. 7, 2023

Related U.S. Application Data
(60) Provisional application No. 63/404,802, filed on Sep. 8, 2022.

(51) Int. Cl. A61B 5/113; A61B 5/00; A61B 5/0205; A61B 5/08

(52) U.S. Cl. CPC A61B 5/113; A61B 5/0205; A61B 5/0816; A61B 5/746

(57) ABSTRACT

Embodiments described herein relate to systems and methods for controlling sensors, receiving sensor data, and evaluating the interrelationship between breath and movement and providing analysis and feedback to the user through a device. The system generates digital instructions and output for an online web application, device hosted application, smart device, or similar system.

### CROSS REFERENCE TO RELATED APPLICATION

The present application claims the benefit of and priority to U.S. Provisional Patent Application No. 63/404,802 entitled METHOD AND SYSTEM FOR RESPIRATION AND MOVEMENT and filed Sep. 8, 2022, the entire contents of which is hereby incorporated by reference.

### FIELD

The present disclosure relates to methods and systems for electrical computers, sensors, digital processing, computer interfaces, online exercise experiences, smart devices, digital classes and environments, monitoring, physiological states, providing exercise instruction, providing exercise guidance, providing exercise feedback, providing wellness guidance, providing visualizations, providing audio feedback, and digital simulation.

### INTRODUCTION

When engaged in activity, such as exercise, the preferred correspondence between respiratory inhalation, pause, exhalation, pause and portions of a motion may provide benefits in performance, stability, endurance, oxygen efficiency and the like. During isotonic exercises (muscle contraction where the length of the muscle changes) with eccentric (muscle elongates) and concentric (muscle contracts) contractions, but also during isometric (muscle length does not change) exercise, aligning the pattern of the breath in relationship to movement may provide benefits. As well, in some forms of activity, or training, alternation of the movement pattern (direction the head turns, which foot strikes the ground) may also have preferred patterns related to the breath. Similarly, the specific timing and/or positioning of inhalation and exhalation prior to/post an anaerobic activity may affect performance. Some individuals find it challenging to align and/or monitor their breathing patterns while engaged in various movements, yoga, exercises, running, martial arts, swimming, meditation, weightlifting, and/or other activities.

### SUMMARY

Embodiments described herein involve automated computer systems and machine learning systems for automatically identifying relationships within input, data or electrical signals representing a user's physical position, moves, and/or movement patterns and breath, or respiratory patterns. Embodiments described herein involve further involve sensors for monitoring respiration, other physiological sensors, audio monitoring, geo-location monitoring, sensors for motion capture, video and image capture and scanning, visual display, streaming overlays, input devices, output devices, image scanning, video scanning, and the like.

Embodiments described herein provide systems, sensors, computer products, and methods for receiving one or more input characterizing a breath and one or more input characterizing a location or movement, computing an interrelationship between the breath and the movement, and providing a breath-move interrelationship evaluation. Embodiments described herein can involve performing measurements for obtaining the input characterizing a breath and one or more input characterizing a location or movement. The input can be sensor data or electrical signals, for example. Embodiments described herein involve executing instructions, by a hardware processor to generate one or more sets of breath-move interrelationship evaluation representation instructions. These instructions can then be interpreted by an output device to activate or trigger one or more outputs to provide breath-move interrelationship feedback and/or guidance for achieving a preferred breath-move interrelation on a user device or output device associated with a user device.

In an aspect, there is provided a device for generating output instructions for a breath-move interrelation evaluation. The device has: a processing system having one or more one hardware processors and one or more memories coupled with the one or more processors programmed with executable instructions to cause the processing system to: transmit control signals to one or more sensors to perform measurements of a user associated with one or more activity of the user; obtain input data from the measurements of the user and contextual metadata, wherein the input data comprises data characterizing a user breath pattern and data characterizing a user movement, wherein the contextual metadata identifies one or more of the user, the activity, an activity type, an activity class, an activity series, and an activity group; compute a set of interrelations between the data characterizing the user breath pattern and the data characterizing the user movement; generate the output instructions for a breath-move interrelation evaluation representation based on the set of interrelations, the breath-move interrelation evaluation being associated with the one or more activity of the user; and transmit the output instructions to provide the breath-move interrelation evaluation representation at a user interface or store the breath-move interrelation evaluation representation in the one or more memories, the output instructions to activate or trigger the user interface to present the breath-move interrelation evaluation representation. The device communicates with the one or more sensors coupled to one or more transmitters, wherein the one or more sensors perform the measurements of the user associated with the one or more activity of the user, the one or more transmitters transmit the measurements to the device.

In some embodiments, the processing system generates a baseline breath-move interrelation evaluation associated with the user or the activity, wherein the processing system generates the output instructions for the breath-move interrelation evaluation representation by comparing the breath-move interrelation evaluation representation to the baseline breath-move interrelation evaluation to determine that the breath-move interrelation evaluation representation varies from the baseline breath-move interrelation evaluation within a threshold.

In some embodiments, the output instructions for the breath-move interrelation evaluation comprise guidance related to cyclical movement.

In some embodiments, the processing system evaluates the set of interrelations between the data characterizing the user breath pattern and the data characterizing the user movement against a preferred interrelation between the data characterizing the user breath pattern and the data characterizing the user movement, wherein the processing system identifies the preferred interrelation using the contextual metadata that identifies the one or more of the user, the activity, the activity type, the activity class, the activity series, and the activity group, wherein the processing system identifies the preferred interrelation using a preferred breath-move interrelation model or a model which comprises one or more preferred breath-move interrelation representation type.

In some embodiments, the activity is selected from the group consisting of sleep, exercise, a wellness activity, work, shopping, watching an event or performance, and gaming. In some embodiments, the activity involves cyclical movement of the user.

In some embodiments, the output instructions to provide the breath-move interrelation evaluation representation at the user interface of the electronic device provides one or more selected from the group of a symbolic visual representing the breath-move interrelation representation as a visual component of the user interface, visual symbol, overlay over a video depicting the user movement, audio feedback, text, graph, summary, message, notification, rating value, rating value within a composite rating, lighting feedback, tactile feedback, vibration feedback, change in music, and heating/cooling feedback.

In some embodiments, the processing system computes the set of interrelations between the data characterizing the user breath pattern and the data characterizing the user movement using a machine learning model for interrelation between breathing patterns and movement.

In some embodiments, the processing system uses a machine learning model comprising one or more preferred breath-move interrelations types to evaluate the set interrelations between the data characterizing the user breath pattern and the data characterizing the user movement.

In some embodiments, the processing system extracts, from the data characterizing the user movement, one or more features selected from the group of an eccentric aspect, a concentric aspect, a stillness threshold aspect, a direction of movement, jerk, cadence, center of gravity, center of pressure, movement duration, movement phase, smoothness, movement associated with a specific body portion and/or limb, an anaerobic threshold aspect, an aerobic threshold, a stillness measure and computes the set of interrelations using the one or more extracted features.

In some embodiments, the processing system uses the data characterizing the user movement to identify an eccentric aspect and associate an inhalation logic, and to identify a concentric aspect and associate an exhalation logic.

In some embodiments, the processing system extracts, from the data characterizing the user breath pattern, one or more features selected from the group of mouth breathing, nose breathing, depth of inhalation, belly expansion, belly tension, consistency, oxygen levels, velocity, rate, volume, coherence, and computes the set of interrelations using the one or more extracted features.

In some embodiments, the processing system identifies one or more of another user, user group, user type, and compares the set of interrelations against a second interrelation associated with one or more of the other user, the user group, the user type, a previously generated interrelation for the user, an exemplary user, a generalized model based on a set of users.

In some embodiments, the processing system evaluates a value associated with a breath-move interrelation evaluation representation and changes content for the user interface with content based on the value by one or more of presenting, removing, unlocking, and customizing, and wherein the content is one or more of a personalization, a feature, a retail offer, a retail experience, a user profile, a user wish list of products or services, a class, a group activity, a workshop, a coaching session, a video, a song, a graphic user interface skin, a performance event, community event, an exercise class, an avatar, an avatar's clothing, an avatar accessory, a conversational interaction, a notification, a pop-up suggestion, an alarm, a badge, a group membership.

In some embodiments, the output instructions to provide the breath-move interrelation evaluation representation provide guidance to shift one or more of the user breath pattern, the user movement pattern to increase correspondence with a preferred breath-move interrelation evaluation.

In some embodiments, the breath-move interrelation evaluation representation is associated with one or more types of breath-move interrelation evaluation representations, wherein the processing system generates the output instructions for the breath-move interrelation evaluation representation by selecting a breath-move interrelation evaluation representation type based on one or more of a user location, a user device, a user group membership, a user device type, a user system type, a user preference, and the activity.

In some embodiments, the processing system is part of one or more selected from the group of an exercise apparatus, exercise platform, a smart mirror, smart phone, a computer, a tablet, a smart exercise device, a fitness tracker, a connected fitness system, a connected audio system, a connected lighting system, a smart exercise device, a component within a connected smart exercise system, a smart mat, a smart watch, a smart sensor, a virtual reality headset, an augmented reality headset, a haptic glove, a haptic garment, a game controller, a hologram projection system, an autostereoscopic projection system, mixed reality devices, virtual reality devices, an augmented reality device, a metaverse headset, a retail platform, a recommendation system, and a social networking community system, gaming platform system, membership system, activity tracking system, machine learning system, a virtual reality environment, an augmented reality environment, a mixed-reality environment, or a combination thereof.

In some embodiments, the processing system communicates with a messaging system to provide the breath-move interrelation evaluation representation through one or more of email, SMS message, MMS message, social media notification, notification message on the user interface.

In some embodiments, the one or more of the sensors is one or more of a camera, a video camera, a microphone type sensor, a heart rate monitor, a breathing monitor, a blood glucose monitor, a humidity sensor, an oximetry sensor, an electronic implant, an EEG, a brain-computer interface, an accelerometer, a restive sensor, a gyroscope, an inertial sensor, a Global Positioning System (GPS) sensor, a Passive Infrared (PIR) sensor, an active infrared sensor, a Microwave (MW) sensor, an area reflective sensor, a lidar sensor, an infrared a spectrometry sensor, an ultrasonic sensor, a vibration sensor, an echolocation sensor, a proximity sensor, a position sensor, an inclinometer sensor, an optical position sensor, a laser displacement sensor, a multimodal sensor, a pressure sensor, an acoustic sensor.

In another aspect, there is provided a non-transitory computer readable medium with instructions stored thereon, that when executed by a hardware processor causes the processor to: transmit control signals to one or more sensors to perform measurements of a user associated with one or more activity of the user; receiving input data characterizing a user breathing pattern from the measurements and input data characterizing a user movement from the measurements, calculating a set of interrelations between the input data characterizing the user breath pattern and the input data characterizing the user movement; generating output instructions for a breath-move interrelation evaluation representation based on the set of interrelations; and transmitting the output instructions to provide the breath-move interrelation evaluation representation at a user interface of an electronic device or storing an indication of the breath-move interrelation evaluation representation in memory.

In some embodiments, the non-transitory computer readable medium has instructions to cause the processor to evaluate the set of interrelations between the breath and movement for a user against a baseline or a preferred interrelation between the breathing pattern and user movement.

In some embodiments, the non-transitory computer readable medium has instructions to provide the breath-move interrelation evaluation representation at the user interface of the electronic device provides one or more selected from the group of visual symbol, overlay over a video depicting the user movement, audio feedback, message, graph, summary, rating value, rating value within a composite rating, lighting feedback, tactile feedback, vibration feedback, change in music, and heating/cooling feedback.

In some embodiments, the non-transitory computer readable medium has instructions for evaluating the interrelations between the user breathing pattern and user movement using a machine learning model associated with interrelation between a breathing pattern and a movement.

In an aspect, there is provided a computer implemented method for generating output instructions for a breath-move interrelation evaluation. The method involves: transmitting control signals to one or more sensors to perform measurements of a user associated with one or more activity of the user and synchronize the one or more one or more sensors performing measurements; receiving, using at least one hardware processor and the one or more sensors to perform measurements, input data that comprises data characterizing a user breath pattern; receiving, using the at least one hardware processor and the one or more sensors to perform measurements, input data that comprises data characterizing a user movement; receiving, using the at least one hardware processor, metadata related to the data characterizing the user breath pattern and the data characterizing the user movement; computing using the at least one hardware processor, a set of interrelations based on the data characterizing the user breath pattern and the data characterizing the user movement; generating, based on the calculated interrelations, the output instructions to provide the breath-move interrelation evaluation; and transmitting the output instructions to provide the breath-move interrelation evaluation representation at a user interface of an electronic device or store the breath-move interrelation evaluation representation in memory.

In some embodiments, the method involves, when receiving, using the hardware processor, the metadata related to the data characterizing the user breath pattern and the data characterizing the user movement, identifying one or more of the activity, activity group, activity type, activity series associated with the data characterizing the user breath pattern and the data characterizing the user movement; identifying a preferred interrelation using the one or more of activity, activity group, activity type, activity series; and evaluating the calculated interrelations against the preferred interrelation.

In some embodiments, the method involves evaluating the calculated interrelations based on a machine learning model which comprises one or more preferred breath-move interrelations type.

In some embodiments, the method involves identifying using the data characterizing the user movement one or more feature of an eccentric aspect, a concentric aspect, a stillness threshold aspect, a direction of movement, jerk, cadence, center of gravity, center of pressure, movement duration, movement phase, smoothness, movement associated with a specific body portion and/or limb, an anaerobic threshold aspect, an aerobic threshold and applying the one or more identified feature as a factor in calculating the interrelations.

In some embodiments, the method involves identifying using the data characterizing the user movement an eccentric aspect and associating an inhalation logic and/or identifying using the data characterizing a movement a concentric aspect and associating an exhalation logic.

In some embodiments, the input data that comprises the data characterizing the user movement comprises a stillness measure.

In some embodiments, the method involves extracting in the data characterizing the user breath pattern one or more features of breathing through mouth, breathing through the nose, depth of inhalation, belly expansion, belly tension, consistency, oxygen levels, velocity, rate, volume, coherence and applying the one or more extracted features as a factor in calculating the set of interrelations.

In some embodiments, the method involves, when receiving, metadata, related to the data characterizing the user breath pattern and the data characterizing the user movement, identifying one or more of another user, user group, user type; and comparing the set of interrelations against a second interrelation associated with one or more of the other user, the user group, the user type, a previously generated interrelation for the user, an exemplary user, a generalized model based on a set of users.

In an aspect, there is provided a non-transitory computer readable medium with instructions stored thereon, that when executed by a hardware processor causes the processor to generate output instructions for a breath-move interrelation evaluation representation, wherein the processor transmits control signals to one or more sensors to perform measurements, the processor generates the output instructions for a breath-move interrelation evaluation by receiving an input characterizing a user breathing pattern, receiving an input characterizing a user movement, calculating a set of interrelations between the user breathing pattern and user movement, and generating the output instructions to provide the breath-move interrelation evaluation representation at a user interface of an electronic device or storing an indication of the breath-move interrelation evaluation representation in memory.

In some embodiments, the user movement is associated with an activity and/or other contextual metadata. In some embodiments, this non-transitory computer readable medium comprises instructions for evaluating the set of interrelations between the breath and movement for a user against a preferred interrelation between the breathing pattern and user movement. In some embodiments, the preferred breath-move interrelationship is based on one or more of general biometric principals, an activity type, a specific activity, a set of activities, the user, metadata associated with the user, a skill level, an instructor or expert breath-move interrelation. In some embodiments, the calculating of interrelations between the user breathing pattern and user movement is evaluated using a data model associated with interrelation between a breathing pattern and a movement.

In some embodiments, the breath-move interrelation evaluation representation provides one or more of visual symbol, overlay over a video depicting the user move, audio feedback, graph, summary, rating value, rating value within a composite rating, lighting feedback, tactile feedback, vibration feedback, change in music, and/or heating/cooling feedback.

In some embodiments, there are provided systems, methods, and/or executable instructions for synchronizing sensor input including the one or more input characterizing a user breathing pattern and one or more input characterizing a user movement.

In an aspect, there is a computer implemented method for generating output instructions for a breath-move interrelation evaluation for a user, the method comprising: transmitting control signals to one or more sensors to perform measurements, receiving, using a hardware processor and one or more sensors to perform measurements, input data that comprises data characterizing a breath pattern; receiving, using a hardware processor and one or more sensors to perform measurements, input data that comprises data characterizing a movement; receiving, using a hardware processor, metadata related to the data characterizing a breath pattern and the data characterizing a movement; calculating using a hardware processor, interrelations based on the data characterizing a breath pattern and the data characterizing a movement; generating, based on the calculated interrelations, the output instructions to provide the breath-move interrelation evaluation.

In some embodiments, this method further comprises evaluating, using a hardware processor, the calculated interrelations against a preferred breath-move interrelation. In some embodiments evaluating the calculated interrelations is based on a model which comprises one or more preferred breath-move interrelations type.

In some embodiments, the method includes identifying using the data characterizing a movement one or more feature of an eccentric aspect, a concentric aspect, a stillness threshold aspect, a direction of movement, jerk, smoothness, movement associated with a specific body portion and/or limb, an anaerobic threshold aspect, a cadence, a center of gravity, a center of pressure, a movement duration, a movement phase, an aerobic threshold and applying the one or more identified feature as a factor in calculating the interrelations. In some embodiments, the method comprises identifying using the data characterizing a movement an eccentric aspect and associating an inhalation logic and/or identifying using the data characterizing a movement a concentric aspect and associating an exhalation logic. In some embodiments, the input data that comprises data characterizing a movement comprises a stillness measure.

In some embodiments, the method further comprises executable instructions for identifying in the data characterizing a breath pattern one or more feature of breathing input through mouth, breathing input through the nose, depth of inhalation, belly expansion, belly tension, consistency, oxygen levels (Sp02), velocity, rate, volume, coherence and applying the one or more identified feature as a factor in calculating the interrelations.

In some embodiments, the method when receiving, metadata related to the data characterizing a breath pattern and the data characterizing a movement, identifies one or more of a user, user group, user type. In some embodiments, the method further comprises analysing the calculated interrelations against a second calculated interrelations associated with one or more of another user, a previously generated set of instructions for the user, an exemplary user, a generalized model based on a set of users.

In some embodiments, when receiving, using a hardware processor, metadata related to the data characterizing a breath pattern and the data characterizing a movement, the method identifies one or more of an activity, activity group, activity type, activity series associated with the data characterizing a breath pattern and the data characterizing a movement. In some embodiments, the one or more of activity, activity group, activity type, activity series is used to identify a preferred interrelations. In some embodiments, the preferred interrelations are one or more of embedded in a specific activity instructions provided to a user device, embedded in a set of specific activity instructions provided to a user device, embedded in an activity type instructions provided to a user device, associated with a specific activity instruction provided to a user device, associated with a set of specific activity instructions provided to a user device, associated with an activity type instructions provided to a user device. In some embodiments, one or more preferred interrelations are a factor when generating output instructions to provide the breath-move interrelation evaluation and/or breath-move interrelation evaluation representation.

In some embodiments, the method further comprises computer implemented output instructions for providing to a user device a representation of the generated breath-move interrelation evaluation. In some embodiments, one or more representation of the breath-move interrelation evaluation are determined, selected, and/or provided based on a model which comprises one or more preferred breath-move interrelation representation type.

The method may provide an input to a machine learning system, receiving an output from a machine learning system. In some embodiments, the method is integrated in a larger system such as a fitness/wellness platform, educational platform, retail platform, membership platform, social network platform or the like.

In an aspect, there is a computer implemented method for providing output instructions for a breath-move interrelation evaluation representation for a user, the method comprising: transmitting control signals to one or more sensors to perform measurements, receiving, using a hardware processor and one or more sensors to perform measurements, input data that comprises data characterizing a breath pattern; receiving, using a hardware processor and one or more sensors to perform measurements, input data that comprises data characterizing a movement; receiving, using a hardware processor, metadata related to one or more of the data characterizing a breath pattern and the data characterizing a movement; calculating using a hardware processor, interrelations based on the data characterizing a breath pattern and the data characterizing a movement; generating, based on the calculated interrelations, the output instructions to provide the breath-move interrelation evaluation; generating, based on the output instructions to provide the breath-move interrelation evaluation, output instructions to provide a representation of the breath-move interrelation evaluation; providing, based on the output instructions, the breath-move interrelation evaluation representation to a user device.

In some embodiments, providing the breath-move interrelation evaluation representation comprises providing a symbolic visual representing the breath-move interrelation representation as a visual component displayed on a user device. In some embodiments, the breath-move interrelation evaluation representation provides one or more of visual symbol, overlay over a video depicting the user move, audio feedback, text, graph, summary, message, notification, rating value, rating value within a composite rating, lighting feedback, tactile feedback, vibration feedback, change in music, heating/cooling feedback. In some embodiments, generating the breath-move interrelation evaluation comprises an assessment comparing the calculated interrelation to one or more preferred interrelation. In some embodiments, the breath-move interrelation evaluation representation comprises providing guidance to shift one or more of the user breath pattern, the user movement pattern to increase correspondence with a preferred breath-move interrelation evaluation.

In some embodiments, the method includes identifying an activity and/or activity type associated with the data characterizing a breath pattern and the data characterizing a movement. This identified activity may be used to evaluate one or more of the breath-move interrelation, the breath-move interrelation evaluation, the breath-move interrelation evaluation representation against a preferred breath-move interrelation model. In some embodiments, the activity of the user effects one or more of the breath-move interrelation evaluation representation provided, the user device on which the breath-move interrelation evaluation representation is provided, and/or the timing of when the breath-move interrelation evaluation representation is provided. In some embodiments, based on the activity associated with the breath-move interrelation evaluation, one or more characteristic of the breath-move interrelation evaluation representation provided changes.

In some embodiments, determining the representation of the breath-move interrelation evaluation to provide is based on a model which comprises one or more preferred breath-move interrelation representation type. In some embodiments, within the breath pattern data one or more of mouth breathing, nose breathing are identified and provided as a factor in the breath-move interrelation calculation.

In some embodiments, the representation is associated with one or more type of representation. In some embodiments, selecting the one or more breath-move interrelation evaluation representation type to provide is based on one or more of a user location, a user device, a user device type, a user group membership, a user system type, a user preference. In some embodiments, the breath-move interrelation evaluation is provided to an exercise platform, or other platform such as a wellness platform, recommendation platform, retail platform, education platform, where the breath-move interrelation representation is generated.

In one aspect, there is a computer system for providing a user device with a breath-move interrelation evaluation representation associated with a specific user breath data and movement data, the system comprising: a communication interface to transmit the specific user breath data and move data, breath-move interrelation evaluation data, breath-move interrelation evaluation representation; one or more non-transitory memory storing a breath-move model; a hardware processor programmed with executable instructions for generating the breath-move interrelation evaluation representation associated with a breath-move interrelation evaluation generator, wherein the hardware processor: transmits control signals to one or more sensors to perform measurements, receives from the one or more sensors to perform measurements input data that comprises data characterizing a breath pattern and data characterizing a movement; receives input data associated with the input data that comprises data characterizing a breath and data characterizing a move comprising contextual metadata; calculates a set of interrelations between the data characterizing a breath and the data characterizing a movement; generates one or more breath-move interrelation evaluation; evaluates providing to a user device a breath-move interrelation evaluation representation; a user device comprising a hardware processor, and an interface to receive the breath-move interrelation evaluation representation; and activate, trigger, or present the breath-move interrelation evaluation representation at a user device output.

In some embodiments, the system provides one or more of a visual symbol, overlay over a video depicting the user move, audio feedback, text, graph, summary, message, notification, rating value, rating value within a composite rating, lighting feedback, tactile feedback, vibration feedback, change in music, heating/cooling feedback. In some embodiments, the system provides a symbolic visual representing the breath-move interrelation representation as a visual component on a user device. In other embodiments, the system provides the breath-move interrelation using audio, tactile, or other techniques, and/or a combination of the visual component displayed on the user device and other means. In some embodiments, providing the breath-move interrelation evaluation representation comprises providing guidance to shift one or more of the user breath pattern, the user movement pattern to increase correspondence with a preferred breath-move interrelation.

Within the system, the user device may be one or more of a smart mirror, smart phone, computer, tablet, smart exercise device, fitness tracker, connected fitness system, connected audio system, a connected lighting system, and the like. In some embodiments, the system further comprises a messaging system to provide a breath-move interrelation evaluation representation to a user through one or more of email, SMS message, MMS message, social media notification, notification message on a user device.

In one aspect, there is a computer system for generating a breath-move interrelation evaluation associated with a specific user's breath data and movement data, the system comprising: a communication interface to transmit the specific user breath data and move data, breath-move interrelation evaluation data, breath-move interrelation evaluation representation; one or more non-transitory memory storing a breath-move model; a hardware processor programmed with executable instructions for generating the breath-move interrelation evaluation representation associated with a breath-move interrelation evaluation, wherein the hardware processor: transmits control signals to one or more sensors to perform measurements, receives from the one or more sensors to perform measurements input data that comprises data characterizing a breath pattern and data characterizing a movement; receives input data associated with the input data characterizing a breath and characterizing a movement comprising contextual metadata; calculates a set of interrelations between the data characterizing a breath and the data characterizing a movement; generates one or more breath-move interrelation evaluation.

In some embodiments, the systems for generating a breath-move interrelation evaluation and/or providing a breath-move interrelation evaluation representation, using the contextual metadata, identifies one or more of a user, an activity, an activity type, a fitness class, a group activity. In some embodiments, these systems further comprise one or more model associated breath-move interrelations, breath-move interrelation evaluations, breath-move interrelation evaluation representations, activities, users, an exercise platform, a recommendation system platform, a retail platform. In some embodiments these system further comprise one or more repository storing one or more of breath-move interrelations, breath-move interrelation evaluations, breath-move interrelation evaluation types, breath-move representations, breath-move representation types.

In some embodiments, one or more of the systems further comprise executable instructions for one or more of providing an input into a system or receiving an output from a system where the system is of one or more of a type exercise system, recommendation system, retail system, social networking community system, gaming platform system, membership system, activity tracking system, machine learning system. In some embodiments, the input data that comprises data characterizing a breath pattern and data characterizing a movement is associated with a user engaging a wellness activity.

In some embodiments, the one or more of the sensors is one or more of a camera, video camera, a heart rate monitor, breathing monitor, earbuds with microphone, a blood glucose monitor, an oximeter, an electronic implant, an EEG, a brain-computer interface, an accelerometer, a gyroscope, an inertial sensor, a GPS, a microphone type sensor, a hologram projection system, an autostereoscopic projection system, virtual reality headset, an augmented reality headset, mixed reality devices, virtual reality devices, an augmented reality device, a metaverse headset, a haptic glove, a game controller, a haptic garment, which may or may not be integrated in other devices.

In some embodiments, ore or more of the systems include a model containing preferred breath-move interrelations, preferred breath-move interrelation evaluations and/or preferred breath-move interrelation evaluation representations. In some embodiments, the system comprises a machine learning component. In some embodiments, the system evaluates providing to a user device a breath-move interrelation evaluation representation; a user device comprising a hardware processor, and an interface to receive the breath-move interrelation evaluation representation; and activate, trigger, or present the breath-move interrelation evaluation representation at a user device output.

This summary does not necessarily describe the entire scope of all aspects of various embodiments described herein. Other aspects, features and advantages can be provided by various embodiments.

### BRIEF DESCRIPTION OF THE DRAWINGS

Embodiments of the disclosure will now be described in conjunction with the accompanying drawings of which:

FIG. 1 shows an example system architecture for generating and/or providing a breath-move interrelation evaluation according to embodiments described herein.

FIG. 2 shows an example system architecture for generating and/or providing a breath-move interrelation evaluation according to embodiments described herein.

FIG. 3 shows an example method of generating a breath-move interrelation evaluation according to embodiments described herein.

FIG. 4 shows an example method associated with receiving breath and move sensor inputs, generating a breath-move interrelation evaluation, and providing breath-move interrelation evaluation representations according to embodiments described herein.

FIG. 5 shows an example method of receiving sensor input and generating and/or providing a breath-move interrelation evaluation according to embodiments described herein.

FIG. 6 shows an example method of generating a breath-move interrelation evaluation according to embodiments described herein.

FIG. 7 shows an aspect related to generating and/or providing a breath-move interrelation evaluation according to embodiments described herein.

FIG. 8 shows an aspect related to generating and/or providing a breath-move interrelation evaluation according to embodiments described herein.

FIG. 9 shows an example method related to generating and/or providing a related to generating and/or providing a breath-move interrelation evaluation according to embodiments described herein.

FIG. 10 shows an example user interface related to generating and/or providing a breath-move interrelation evaluation according to embodiments described herein.

FIG. 11 shows an example user interface related to generating and/or providing a breath-move interrelation evaluation according to embodiments described herein.

FIG. 12 shows an example user interface related to generating and/or providing a breath-move interrelation evaluation according to embodiments described herein.

FIG. 13 shows an example user interface related to generating and/or providing a breath-move interrelation evaluation according to embodiments described herein.

### DETAILED DESCRIPTION

The methods, devices and systems involve a hardware processor having executable instructions to provide one or more calculated breath-move interrelation evaluations based on one or more inputs characterizing a breath and a move for a user. The input can be sensor data or electrical signals, for example. The input data can be captured in real-time or near real-time. Ventilation is another term used to refer to characteristics of breath and breathing. In some embodiments, the method and system can involve one or more sensors performing measurements of a user and a processing system for obtaining the inputs characterizing a breath and one or more input characterizing a location or movement. In some embodiments, the method and system provide a series of breath-move interrelation evaluation calculations which are associated with one or more activity of the user over a time duration. In some embodiments, one or more sensors can perform measurements of a user for an activity. The methods, devices and systems can use the measurements to derive or obtain input data for generating the breath-move interrelation evaluation. In some embodiments, one or more controllers can be used to obtain measurements of the user for the activity. The methods, devices and systems can use different hardware devices to perform measurements. A sensor can be an device that detects or measures a physical property, records the detections or measurements, or transmits the detections or measurements. A sensor is a device that responds to a physical stimulus and generates a resulting measurement. A sensor can be a device, machine, or subsystem that detects events or changes in its environment, produces an output signal associated with sensing physical properties, and sends the information to other electronics, such as a hardware processor. A controller can be an electronic device (e.g. as part of a control system) that generates control signals as output to control actions of the device or another device. For example, a controller can generate control signals with code that controls operations of a processor or peripheral device, actuate components of a device, and so on. A controller can be a chip, card, an application, or hardware device. A controller can manage and connect devices, or direct the flow of data between devices to provide an interface and manage communication. For example, a controller can be an interface component between a central processing system and a device being controlled. A controller can be a type of input device that generates and transmits control commands to control operations of a sensor, a computer, a component, or other device. There are different types of controllers. Controllers automatically control products, embedded systems, and devices using control commands. A controller can couple to one or more processors, memory and programmable input/output peripherals. Examples of sensors and controllers are disclosed herein.

In some embodiments, the breath-move interrelation evaluation provides feedback on the interrelation of a breath pattern and a movement pattern in accordance with activity models. In some embodiments, the breath-move interrelation evaluation is associated with and/or compared to one or more preferred breath-move interrelation patterns for the motion, activity, class, activity intensity, or wellness activity. In embodiments, guidance is provided when the user's breath-move interrelationship is evaluated to be, based on a measured threshold, out of a specific range of synchronicity or coherence with a preferred breath-move interrelationship. Embodiments can involve different ways of providing feedback and/or guidance such as, for example, one or more of visual feedback, guidance, video, texts, charts, changed display content, overlays, symbols, message, graph, summary, rating value, rating value within a composite rating, color-coded indicators, numeric indicators, flashing the display graphics, audio feedback, lighting feedback, vibration feedback, heating/cooling feedback, scented feedback, and the like. The feedback on the breath-move interrelation evaluation can provide a stimulus for the user.

Embodiments described herein transmit signals to one or more sensors to preform measurements and receive an input characterizing a user breathing pattern and a user movement or location. Embodiments described herein can involve using one or more sensors to preform measurements and receiving, from the measurements, input characterizing a user breathing pattern and a user movement or location. Calculated breath-move interrelation evaluations are transmitted to and interpreted by an application and/or output device to activate, display or trigger one or more breath-move interrelation evaluations, or provide the user with one or more breath-move interrelation evaluation representation. In an aspect, embodiments described herein provide, to a user, a breath-move interrelation evaluation (BMIE), based on input characterizing a user breathing pattern and a user movement or location, also referred to as a breath-move evaluation. As the interrelation of breath and movement involves change over a duration, a BMIE may be contextualized within a series of BMIE, also referred to as a BMIE map. A BMIE map may be associated with an activity or activity type. Likewise, in some embodiments, breath-move interrelations are contextualized within a series in order to generate a breath-move evaluation.

A breath-move interrelation evaluation can be a measure, prediction or estimate of one or more relationships within breath data and movement data. A breath-move interrelation evaluation representation may be defined as computer executable instructions to generate a representation of the breath-move interrelation evaluation such as an indicator, depiction or guide for the predicted or estimated relationship(s) within breath data and movement data displayed at an interface or otherwise provided to the user, such as by audio, visual, or tactile feedback. The breath-move interrelation evaluation representation may relate to one or more breath-move interrelation evaluations or a series of breath-move interrelation evaluations.

Embodiments described herein can provide improved methods and systems for user feedback on the interrelation of their breath and movement which can improve physical performance, endurance, mood, and/or engagement with a wellness activity.

The feedback can be audio/visual feedback or tactile feedback, for example. Breath-move interrelation evaluations may be represented using visual feedback, guidance, video, texts, charts, changed display content, overlays, symbols, message, graph, summary, rating value, rating value within a composite rating, color-coded indicators, numeric indicators, flashing the display graphics, audio feedback, lighting feedback, vibration feedback, heating/cooling feedback, scented feedback, messages, notifications, and the like.

In some embodiments, the breath-move evaluation is evaluated, assessed, or estimated based on data and data models associated with the user, another user, a model of a user with specific characteristics, an activity, a model of an activity with specific characteristics, a skill level, metadata associated with activity instruction, and the like. Embodiments provide improved evaluation and processing of sensor data to increase the accuracy of feedback and/or to increase the accuracy of detected relationships within the sensor data. Embodiments described herein can generate, use, and/or train models for breath-move interrelation evaluations. Models can be computer programs or code representations of machine learning or artificial intelligence processes that may be trained with datasets to obtain output results for the breath-move evaluations. The breath-move evaluations can provide insights of relationships between breath data and movement data as estimations or predictions.

In some embodiments, the breath-move interrelation is an output to a wellness recommendation system. In some embodiments, the breath-move interrelation is an input to a wellness recommendation system. In some embodiments, the breath-move interrelation is provided through a smart mirror type device, a smart exercise bike, a smart fitness watch, a smart yoga mat, a smart connected system, or the like. In some embodiments, the breath-move interrelation is provided through a virtual reality environment, an augmented reality environment, a mixed-reality environment, or a combination. In some embodiments, the breath-move interrelation is provided within the context of a real-time, near real-time, and/or recorded and streamed, guided wellness activity.

In some embodiments, points or wellness scores are associated with characteristics of the breath-move interrelation evaluation and/or the BMIE is a factor in the calculation of these points and/or scores. In some embodiments, the system provides a “redo” option and/or tutorial for an activity segment where the breath-move interrelation varies from a preferred breath-move interrelation.

Embodiments relate to methods and systems with non-transitory memory storing instructions and data records for breath-move interrelation characterization, user characterization, and/or activity characterization. Embodiments relate to generating and providing a user with feedback and/or other information based on a calculated breath-move interrelation. This feedback and/or other information may include real-time or near real-time feedback related to specific breath, movement, and/or breath-move interrelation characteristics, summary feedback, guidance or instruction for achieving a preferred breath-move interrelation, assigning points to a breath-move interrelation, post-activity feedback related to specific breath, movement, and/or breath-move interrelation characteristics, summary feedback, guidance or instruction for achieving a preferred breath-move interrelation, assigning points to a breath-move interrelation, a combination, or the like.

Breath-move interrelation evaluation representations may involve various media types and combinations of various media types. Example media types include video, interactive presentation, game, image, hologram image projection, autostereoscopic image projection, audio, text, spoken word, guided conversation, email, SMS (short message service) message, MMS (multimedia messaging service) message, music, and/or interactive simulation.

In some embodiments, a breath-move interrelation evaluation may include generating and/or providing executable instructions to present, remove, unlock, or customize one or more of a personalization, a feature, a retail offer, a retail experience, a user profile, a user wish list of products or services, a class, a group activity, a workshop, a coaching session, a video, a song, a graphic user interface skin, a performance event, community event, an exercise class, an avatar, an avatar's clothing, an avatar accessory, a conversational interaction, a notification, a pop-up suggestion, an alarm, a badge, a group membership. In some embodiments, the BMIE, or a value calculated based on the BMIE, is an input to an exercise platform, retail platform, social media community platform, augment reality platform, virtual reality platform, mixed-reality platform or combination thereof.

A breath-move evaluation representation may be provided in different ways and/or using different devices, including, for example, one or more of a web application, an application installed on a user device, a smart mirror device, a connected music system, a connected exercise mat, a connected smell diffuser device, a virtual reality headset, an augmented reality headset, a metaverse headset, a haptic glove, a game controller, a haptic garment, a retail application, a coaching application, a fitness class or studio application, an email system, a text message system, notification system, augmented reality environment, simulated reality environment, virtual reality environment, a game environment, a metaverse or virtual environment. A breath-move evaluation representation may be provided in a combination of different ways and/or different devices. Breath-move evaluation representations may be evaluated automatically by one or more hardware processers based on their capacity to engage a user.

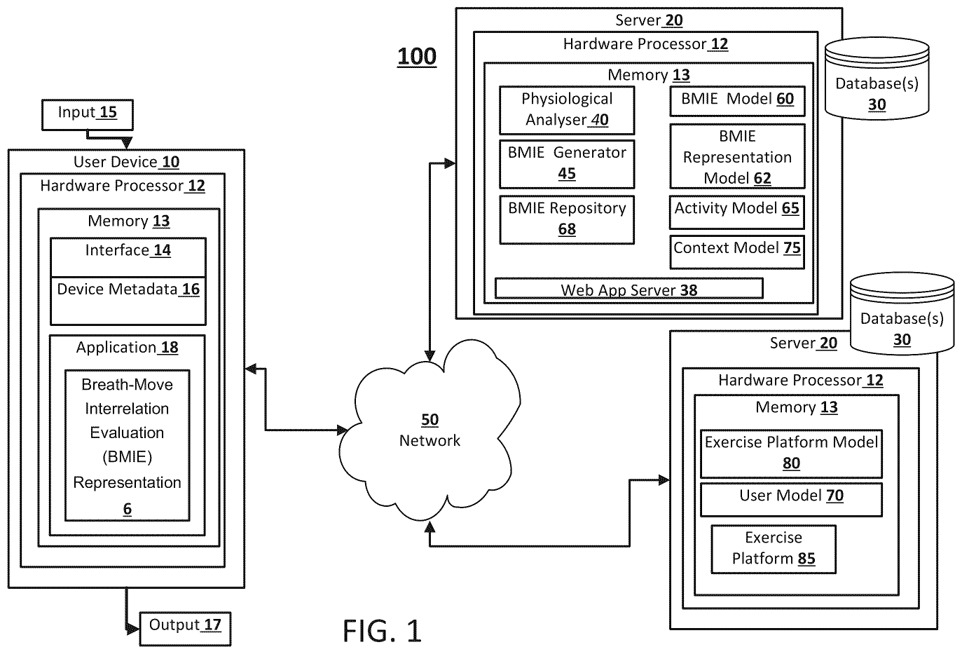

Turning to FIG. 1, there is shown an embodiment of breath-move evaluation system 100 that may generate and/or provide one or more breath-move interrelation evaluation based on an input characterizing a user breathing pattern and an input characterizing a user location or movement, breath-move evaluation system 100 may implement operations of the methods described herein. Breath-move evaluation system 100 has hardware servers 20, databases 30 stored on non-transitory memory, a network 50, and user devices 10. Servers 20 have hardware processors 12 that are communicatively coupled to databases 30 stored on the non-transitory memory and are operable to access data stored on databases 30. Servers 20 are further communicatively coupled to user devices 10 via network 50 (such as the Internet). Thus, data may be transferred between servers 20 and user devices 10 by transmitting the data using network 50. The user devices 10 include non-transitory computer readable storage medium storing instructions to configure one or more hardware processors 12 to provide an interface 14 for collecting data and exchanging data and commands with other components of the system 100. The user devices 10 have one or more network interfaces to communicate with network 50 and exchange data with other components of the system 100. The servers 20 may also have a network interface to communicate with network 50 and exchange data with other components of the system 100.

A number of users of breath-move evaluation system 100 may use user devices 10 to exchange data and commands with servers 20 in manners described in further detail below. For simplicity of illustration, only one user device 10 is shown in FIG. 1, however, breath-move evaluation based on an input characterizing a user breath pattern and a user movement and/or location system 100 can include multiple user devices 10, or even a single user device 10. The user devices 10 may be the same or different types of devices. The breath-move evaluation system 100 is not limited to a particular configuration and different combinations of components can be used for different embodiments. Furthermore, while breath-move evaluation system 100 shows two servers 20 and two databases 30 as an illustrative example related to generating and/or providing a breath-move interrelation evaluation, system 100 extends to different numbers of (and configurations of) servers 20 and databases 30 (such as a single server communicatively coupled to a single database). The servers 20 can be the same or different types of devices.

The user device 10 has at least one hardware processor 12, a data storage device 13 (including volatile memory or non-volatile memory or other data storage elements or a combination thereof), and at least one communication or network interface 14. The user device 10 components may be connected in various ways including directly coupled or indirectly coupled via a network 50. The user device 10 is configured to carry out the operations of methods described herein.

The user device 10 may be a smart exercise device, or a component within a connected smart exercise system. Types of smart exercise devices include smart mirror device, smart treadmill device, smart stationary bicycle device, smart home gym device, smart weight device, smart weightlifting device, smart bicycle device, smart exercise mat device, smart rower device, smart elliptical device, smart vertical climber, smart swim machine, smart boxing gym, smart boxing bag, smart boxing dummy, smart grappling dummy, smart dance studio, smart dance floor, smart dance barre, smart balance board, smart slide board, smart spin board, smart ski trainer, smart trampoline, or smart vibration platform. Additional smart devices that can be used in such a system include a connected audio music system, a connected lighting system. System users may incorporate equipment and/or athletic apparel and equipment in the course of their activity, for example, running shoes, jump ropes, weights, bicycles, swimming pools, mats, and the like. User in such systems may also input data and/or receive breath-move evaluations through different devices such as a camera, video camera, earbuds with microphone, heart rate monitor, heart rate variability monitor, breathing monitor, a blood glucose monitor, an oximeter, an electronic implant, an EEG, a brain-computer interface, an accelerometer, a gyroscope, an inertial sensor, a GPS, a microphone type sensor, a hologram projection system, an autostereoscopic projection system, virtual reality headset, an augmented reality headset, mixed reality devices, virtual reality devices, an augmented reality device, a metaverse headset, a haptic glove, a game controller, a haptic garment, which may or may not be integrated in other devices.

Each hardware processor 12 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof. Memory 13 may include a suitable combination of any type of computer memory that is located either internally or externally such as.

Each network interface 14 enables computing device 10 to communicate with other components, to exchange data with other components, to access and connect to network resources, to serve applications, and perform other computing applications by connecting to a network 50 (or multiple networks) capable of carrying data. The communication or network interface 14 can enable user device 10 to interconnect with one or more input devices, such as a keyboard, mouse, camera, touch screen, sensors and a microphone, or with one or more output devices such as a display screen and a speaker.

The memory 13 can store device metadata 16 which can include available metadata for factors such as memory, processor speed, touch screen, resolution, camera, video camera, processor, device location, haptic input/output devices, augmented reality glasses, virtual reality headsets. The system 100 can determine device capacity for a breath-move interrelation evaluation representation type by evaluating the device metadata 16, for example.

According to some embodiments, user device 10 is a mobile device such as a smartphone, although in other embodiments user device 10 may be any other suitable device that may be operated and interfaced with by a user. For example, user device 10 may comprise a laptop, a personal computer, an interactive kiosk device, immersive hardware device, smart watch, smart mirror or a tablet device. User device 10, may include multiple types of user devices and may include a combination of devices such as smart phones, smart watches, computers, tablet devices, within system 100.

User device 10 receives (or couples to) one or more input 15 characterizing a breath and one or more input characterizing a location or movement, computing an interrelationship between the breath and the movement, and providing a breath-move interrelationship evaluation. The input 15 can be sensor data or electrical signals, for example. In some embodiments, the input 15 can include sensors (or other devices) for performing measurements for obtaining sensor data or electrical signals characterizing a breath, a location, and/or movement.

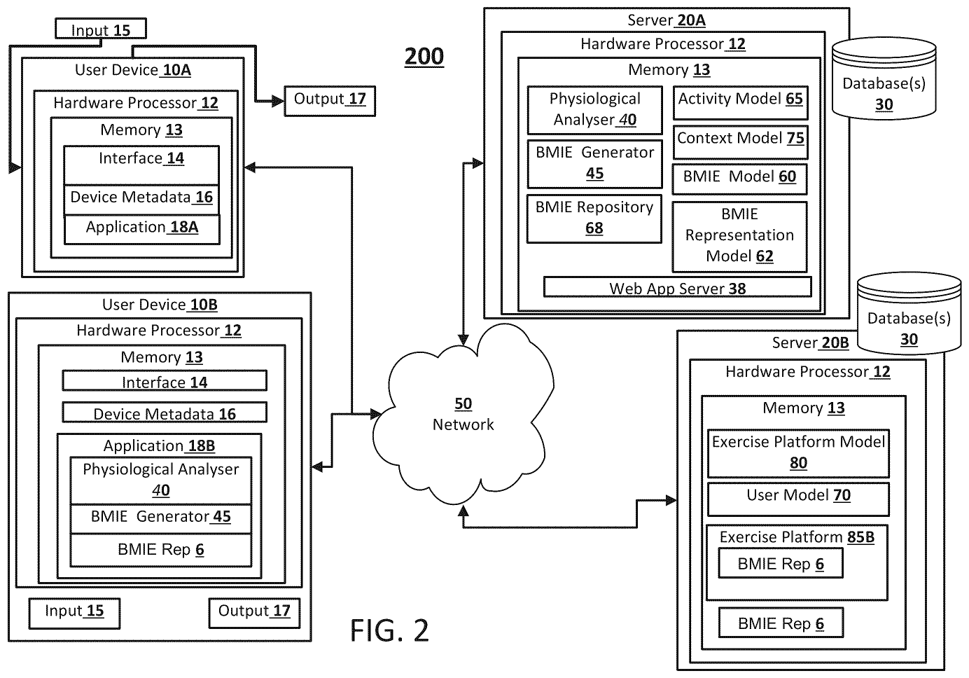

In FIG. 1 the example server architecture includes a server 20 BMIE generator 45 providing a BMIE representation 6 in application 18 to user device 10. In other example architectures, similar functionality is by server 20, web app server 38, or online retail 85 (FIG. 2). Executable instructions or code components such as physiological analyser 40, BMIE generator 45, BMIE model 60, BMIE representation model 62, activity model 65, and BMIE repository 68 may be installed on more than one server 20 within system 100. Server 20 can generate, use, and/or train BMIE models 60 for breath-move evaluations and BMIE representation models 62 for BMIE representations. Models can be computer programs or code representations of machine learning or artificial intelligence processes that may be trained with datasets to obtain output results for the BMIEs and BMIE representations. The BMIEs can provide estimations or predictions of relationships between breath data and movement data, and the BMIEs can provide indicators of the relationships between breath data and movement data. In some example architectures, physiological analyser 40, BMIE generator 45 may be installed on user device 10. In some embodiments, one or more of physiological analyser 40, BMIE generator 45, BMIE model 60, BMIE representation model 62, BMIE repository 68, activity Model 65, context model 75 are combined. In some embodiments there is an interrelation model which contains underlying values which may be used to generate a BMIE and/or be associated with the BMIE model.

The server 20 has at least one hardware processor 12, a data storage device 13 (including volatile memory or non-volatile memory or other data storage elements or a combination thereof), and at least one communication or network interface. The server 20 components may be connected in various ways including directly coupled or indirectly coupled via a network 50. The server 20 is configured to carry out the operations of methods described herein.

User device 10 includes input and output capacity (via network interface 14 or I/O interface), a hardware processor 12, and computer-readable medium or memory 13 such as non-transitory computer memory storing computer program code. Input device 15 may be integrated within user device 10 or connected in various ways including directly coupled or indirectly coupled via a network 50. The input device 15 can perform verifications and scans. For example, the input device 15 can include (or couple to) one or more sensors that can measure breathing patterns, movement, location, heartrate, codes, and IDs relating to a user, activity, or its environment or context. The input device 15 can perform measurements for obtaining input data. A hardware processor 12 can receive input data from the sensors and inputs 15. Similarly, output device 17 may be integrated within user device 10 or connected in various ways including directly coupled or indirectly coupled via a network 50. The output device 17 can activate, trigger, or present one or more BMIE over a time duration. For example, output device 17 can activate or trigger audio associated with a BMIE at a speaker device. As another example, output device 17 can present a visual indicator associated with a BMIE and/or a visual BMIE representation at a display device. As a further example, output device 17 can provide a virtual reality headset experience to enable a virtual experience type BMIE representation.

The BMIE may involve different types of devices to generate different types of discernible effects to provide a multi-sensory BMIE experience. In some embodiments, multiple BMIE can be provided over a time period. For example, a first BMIE can be provided at a first time, a second BMIE can be provided at a second time, and so on. In some embodiments, BMIE representations can be provided simultaneously at a first time, and another BMIE representation can be provided a second time, and so on. In some embodiments, selected BMIE may be stored and provided at a later time. An example of this is a graphical user interface showing the user a series or collection of BMIE representations associated with an activity they are performing, have recently completed, or have completed in the past. User device 10 may be coupled with more than one input device 15, more than one output device 17, and more than one of both input device 15 and output device 17. A single device may contain input device 15 and output device 17 functionality, a simple example of this would be a connected headset with integrated microphone.

Accordingly, FIG. 1 depicts different example devices for generating output instructions for BMIEs. A device can have a processing system having one or more one hardware processors 12 and one or more memories 13 coupled with the one or more processors 12 programmed with executable instructions to cause the processing system to perform operations. For example, the processing system can transmit control signals to one or more sensors (e.g. input 15) to perform measurements of a user associated with one or more activity of the user. The processing system can obtain input data from the measurements of the user and contextual metadata. The input data can involve data characterizing a user breath pattern and data characterizing a user movement. The contextual metadata identifies one or more of the user, the activity, an activity type, an activity class, an activity series, and an activity group. The processing system can compute a set of interrelations between the data characterizing the user breath pattern and the data characterizing the user movement. The processing system can process the data characterizing the user breath pattern and the data characterizing the user movement to compute BMIEs to indicate how the data characterizing the user breath pattern relates to the data characterizing the user movement. This can involve using one or more machine learning models to detect patterns in the data characterizing the user breath pattern and the data characterizing the user movement to detect relationships between the data characterizing the user breath pattern and the data characterizing the user movement.

The processing system can generate the output instructions for a BMIE representation based on the set of interrelations. For example, server 20 can generate the output instructions in some embodiments. As another example, application 18 at user device 10 can generate the output instructions in some embodiments. The BMIE can be associated with the one or more activity of the user. The processing system can transmit the output instructions to provide the BMIE representation at a user interface 14 or store the BMIE representation in the one or more memories 13. The output instructions to activate or trigger the user interface 14 to present the BMIE representation. The device 10 or server 20 communicates with or integrates controllers or sensors coupled to one or more transmitters. The one or more sensors perform the measurements of the user associated with the one or more activity of the user. The controllers can control the sensors to obtain the measurements. The one or more transmitters transmit the measurements to the device 10 or server 20. In some embodiments, the one or more of the sensors is one or more of a camera, a video camera, a microphone type sensor, a heart rate monitor, a breathing monitor, a blood glucose monitor, a humidity sensor, an oximetry sensor, an electronic implant, an EEG, a brain-computer interface, an accelerometer, a restive sensor, a gyroscope, an inertial sensor, a Global Positioning System (GPS) sensor, a Passive Infrared (PIR) sensor, an active infrared sensor, a Microwave (MW) sensor, an area reflective sensor, a lidar sensor, an infrared a spectrometry sensor, an ultrasonic sensor, a vibration sensor, an echolocation sensor, a proximity sensor, a position sensor, an inclinometer sensor, an optical position sensor, a laser displacement sensor, a multimodal sensor, a pressure sensor, an acoustic sensor.

In some embodiments, the processing system generates a baseline BMIE associated with the user or the activity. The processing system generates the output instructions for the BMIE representation by comparing the breath-move interrelation evaluation representation to the baseline BMIE to determine that the breath-move interrelation evaluation representation varies from the baseline BMIE within a threshold.

In some embodiments, the output instructions for the BMIE involve control commands to indicate or output guidance related to cyclical movement at application 18.

In some embodiments, the processing system evaluates the set of interrelations between the data characterizing the user breath pattern and the data characterizing the user movement against a preferred interrelation between the data characterizing the user breath pattern and the data characterizing the user movement. The processing system identifies the preferred interrelation using the contextual metadata that identifies the one or more of the user, the activity, the activity type, the activity class, the activity series, and the activity group. The processing system identifies the preferred interrelation using a preferred breath-move interrelation model or a model which comprises one or more preferred breath-move interrelation representation type.

In some embodiments, the activity is selected from the group consisting of sleep, exercise, a wellness activity, work, shopping, watching an event or performance, and gaming. In some embodiments, the activity involves cyclical movement of the user, and the BMIE can help align strokes or strides or other movements of the user based on a cyclical pattern.

In some embodiments, the output instructions to provide the BMIE representation at the user interface 13 of the electronic device 10 provides one or more selected from the group of a symbolic visual representing the breath-move interrelation representation as a visual component of the user interface, visual symbol, overlay over a video depicting the user movement, audio feedback, text, graph, summary, message, notification, rating value, rating value within a composite rating, lighting feedback, tactile feedback, vibration feedback, change in music, and heating/cooling feedback.

Accordingly, the BMIE representation involves tangible effects based on the updates.

In some embodiments, the processing system computes the set of interrelations between the data characterizing the user breath pattern and the data characterizing the user movement using a machine learning model for interrelation between breathing patterns and movement.

In some embodiments, the processing system uses one or more machine learning models comprising one or more preferred breath-move interrelations types to evaluate the set interrelations between the data characterizing the user breath pattern and the data characterizing the user movement to identify patterns and relationships in the data sets.

In some embodiments, the processing system extracts, from the data characterizing the user movement, one or more features selected from the group of an eccentric aspect, a concentric aspect, a stillness threshold aspect, a direction of movement, jerk, cadence, center of gravity, center of pressure, movement duration, movement phase, smoothness, movement associated with a specific body portion and/or limb, an anaerobic threshold aspect, an aerobic threshold, a stillness measure and computes the set of interrelations using the one or more extracted features. The processing system can identify relations between the features extracted from the data.

In some embodiments, the processing system uses the data characterizing the user movement to identify an eccentric aspect and associate an inhalation logic, and to identify a concentric aspect and associate an exhalation logic.

In some embodiments, the processing system extracts, from the data characterizing the user breath pattern, one or more features selected from the group of mouth breathing, nose breathing, depth of inhalation, belly expansion, belly tension, consistency, oxygen levels, velocity, rate, volume, coherence, and computes the set of interrelations using the one or more extracted features.

In some embodiments, the processing system identifies one or more of another user, user group, user type, and compares the set of interrelations against a second interrelation associated with one or more of the other user, the user group, the user type, a previously generated interrelation for the user, an exemplary user, a generalized model based on a set of users.

In some embodiments, the processing system evaluates a value associated with a breath-move interrelation evaluation representation and changes content for the user interface with content based on the value by one or more of presenting, removing, unlocking, and customizing, and wherein the content is one or more of a personalization, a feature, a retail offer, a retail experience, a user profile, a user wish list of products or services, a class, a group activity, a workshop, a coaching session, a video, a song, a graphic user interface skin, a performance event, community event, an exercise class, an avatar, an avatar's clothing, an avatar accessory, a conversational interaction, a notification, a pop-up suggestion, an alarm, a badge, a group membership.

In some embodiments, the output instructions to provide the BMIE representation provide guidance to shift one or more of the user breath pattern, the user movement pattern to increase correspondence with a preferred BMIE, or a baseline BMIE.

In some embodiments, the BMIE representation is associated with one or more types of breath-move interrelation evaluation representations, wherein the processing system generates the output instructions for the breath-move interrelation evaluation representation by selecting a breath-move interrelation evaluation representation type based on one or more of a user location, a user device, a user group membership, a user device type, a user system type, a user preference, and the activity.

In some embodiments, the processing system is part of one or more selected from the group of an exercise apparatus, exercise platform, a smart mirror, smart phone, a computer, a tablet, a smart exercise device, a fitness tracker, a connected fitness system, a connected audio system, a connected lighting system, a smart exercise device, a component within a connected smart exercise system, a smart mat, a smart watch, a smart sensor, a virtual reality headset, an augmented reality headset, a haptic glove, a haptic garment, a game controller, a hologram projection system, an autostereoscopic projection system, mixed reality devices, virtual reality devices, an augmented reality device, a metaverse headset, a retail platform, a recommendation system, and a social networking community system, gaming platform system, membership system, activity tracking system, machine learning system, a virtual reality environment, an augmented reality environment, a mixed-reality environment, or a combination thereof.

In some embodiments, the processing system communicates with a messaging system to provide the BMIE representation through one or more of email, SMS message, MMS message, social media notification, notification message on the user interface.

In FIG. 1, there is shown an embodiment of a user device 10 where the application 18 includes executable instructions displaying information related to providing BMIE representations 6. For example, in an embodiment, application 18 may be an application providing streaming exercise content displayed on a smart mirror user device 10 which includes executable instructions related to the generating and/or providing of BMIE. Application 18 may be one or more application provided by user device 10. For example, one application 18 program may provide functionality related to capturing sensor data related to a user activity and one application 18 may provide functionality related to providing a BMIE. Application 18 may provide a web browser type program, or other application that enables a user to access BMIE representation 6 stored on server 20B as shown in FIG. 2.

In some embodiments, the function of databases 30 may be implemented by servers 20 with non-transitory storage devices or memory. In other words, servers 20 may store the user data located on databases 30 within internal memory and may additionally perform any of the processing of data described herein. However, in the embodiment of FIG. 1, servers 20 are configured to remotely access the contents of databases 30, or store data on databases 30, when required.

Turning to FIG. 2, there is shown another embodiment of a user device 10A where the application 18A includes executable instructions for accessing breath-move interrelation evaluation representations on server 20B. As shown in FIG. 2, breath-move interrelation evaluation representation 6 can be provided in memory 13 on server 20B and/or breath-move interrelation evaluation representations 6 may be provided in exercise platform 85B, or another component, providing information concerning BMIEs. As shown in FIG. 2, with example user device 10B, application 18B can also provide functionality associated with physiological analyser 40, BMIE generator 45, to provide BMIE representation 6 within memory 13 of a user device 10B.

In some embodiments, models include BMIE model 60, BMIE representation model 62, activity model 65, context model 75, user model 70, exercise platform model 80. These models may be stored in memory 13, database 30. In some embodiments, activity model 65 is integrated in BMIE repository 68 and/or exercise platform model 80 is integrated in exercise platform 85. Models are encoded instructions or programs that are executable by hardware processors to recognize patterns in data or make predictions.

The breath-move evaluation system 200 evaluates user breath patterns, location and/or movement (captured through sensors/input 15 and received as input data) to generate and/or provide a BMIE, and in conjunction with physiological analyser 40 and/or BMIE generator 45, and in some embodiments, activity model 65, and/or BMIE repository 68 evaluates the type of user, activity, and/or BMIE characteristics and generates the context aware BMIE. In some embodiments, the device metadata data 16 and/or application 18 functionality shown on user device 10 is integrated in exercise platform 85.

In some embodiments, the BMIE representation is generated as executable instructions stored within application 18. In some embodiments the BMIE representation is streamed to user device 10 through network 50. The user device 10 and/or output device 17 may be a device such as a smart home peripheral device, smart exercise mirror, or a virtual reality connected device.

The breath-move evaluation system 200 has non-transitory memory storing data records, context data, user breath pattern data, user location and/or movement data, user data, activity data, and additional metadata received from a plurality of channels, at servers 20 and databases 30. For example, the data records can involve a wide range of data related to users, user physiological patterns, user types, user activity, user schedules, user regions, user purchases, user context, activity types, user device capacity, feel-states, product descriptions, product types, product sizing, product availability, retail regions, retail offers, retail promotions, device metadata, and the like. The data involves structured data, unstructured data, metadata, text, numeric values, images, biometric data, physiological data, activity data, renderings based on images, video, audio, sensor data, and so on.

For example, the contextual data includes data that pertains to the context for the user activity associated with the breath, movement, and/or location, sensor inputs, generating a breath-move interrelation, BMIE, and/or BMIE representation. In some embodiments, contextual data contains data identifying qualities such as activities suited to real-time BMIE or summary post activity BMIE based on the activity type/location, specific contextual user data, user classification metadata, user current activity, user historical activity, current lighting, lighting history, specific contextual retail activity, categories of retail activity, specific contextual activity/movement profile data, categories of activity/movement profile data, specific contextual, specific feel state data, categories of feel state data, data, and so on. In some embodiments, input device 15 provides one or more element of the context data.

There will now be described methods for generating BMIEs for a user device 10 based on receiving an input (sensor and/or other input device) characterizing a user breath pattern, location, and/or movement, and activity models, and providing the BMIE to the user. The methods can involve transmitting control signals to one or more sensors to perform measurements (e.g., using sensors and cameras) relating to a user and user activity, and/or an environment. The methods can involve triggering, activating, or presenting one or more BM IE and/or BM IE representations over a time duration to provide discernible effects, including actuation of physical hardware components. The methods can involve providing an indicator of a BMIE associated with a representation associated with the video or image of a user displayed. The methods can involve receiving from a user one or more input and generating and/or providing one or more BMIE. Accordingly, the methods involve computer hardware and physical equipment to perform measurements for the input data, and/or provide discernible output breath-move evaluations.

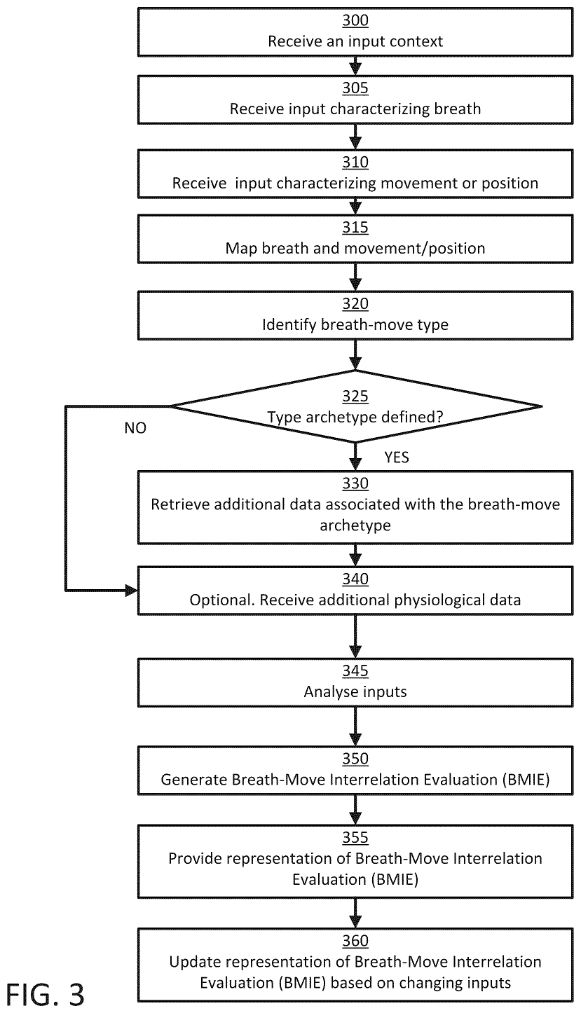

Methods, and aspects or operations of methods are shown generally in FIGS. 3-9 which show diagrams of the steps that may be taken to provide and generate a breath-move interrelation evaluation based on an input charactering a user's breath and an input characterizing a user's movement and/or lack of movement. The steps shown in FIGS. 3-9 are exemplary in nature, and, in various embodiments, the order of the steps may be changed, and steps may be omitted and/or added without departing from the scope of the disclosure. Methods can perform different combinations of operations described herein to provide or generate breath-move interrelation evaluations and representations associated with breath-move interrelation evaluations.

Turning to FIG. 3, in accordance with some embodiments, there is a method of generating breath-move interrelation evaluation based on an input characterizing a breath pattern and/or qualities associated with the breath of the user and an input characterizing a movement and/or qualities associated with the movement of the user. Methods associated with embodiments, involve transmitting control signals to one or more sensors to perform measurements, and receiving, using a hardware processor and one or more sensors to perform measurements, input data that includes data characterizing a breath pattern and data characterizing a movement.

The process may be initiated from a number of different contexts such as participating within a smart mirror based activity (exercise class, training session, concert), a workout or wellness activity performed by an individual, a virtual reality context, a wellness recommendation system, an online social media environment, a retail environment, and/or using an application specifically for evaluating breath-move interrelation evaluations and receiving representations of these evaluations and/or guidance based on these evaluations. The process may be triggered by an individual engaging in a personal workout on their own, seated meditation practice, running, swimming, paragliding, kayaking, engaging in a team sport or group fitness activity, and the like. The process may be initiated based on a user interaction, as part of a larger process, and/or as a default system behaviour. The method of FIG. 3 is applicable to a specific user, a community of users, a class instructor, an educator, an influencer, a simulated representation of an individual user, a simulated representation of a community of users, and the like.

This method is applicable to generating templates for breath-move interrelation evaluations, representations of breath-move interrelation evaluations, guidance and/or recommendations based on breath-move interrelation evaluations, generalized models of breath-move interrelations and specific models of breath-move interrelations based on activity, user profile, and/or other factors, and a combination of templates, models, guidance and/or recommendations. Breath-move evaluations are an analysis of a specific breath characterization and movement characterization based on sensor input from a specific user, or specific group of users. This sensor data input may be provided as recorded data, streamed data, real-time data, near-real-time or a combination thereof. In some embodiments, machine learning, and/or other forms of predictive logic may be used to model and/or extrapolate probable input values when there are gaps in the sensor data input stream.

Receive an input context 300 comprises executable instructions which when executed by a hardware processor causes the processor to receive information, data and metadata, associated with the user sensor data. In some embodiments, the input is received by user device 10 input 15. In some embodiments, the input is previously recorded and stored on user device 10 memory 13 or elsewhere in the system. This input is then received and evaluated by physiological analyser 40 on server 20. In some embodiments, physiological analyser 40 may be provided on user device 10.

Input context can include a token, ID, machine executable code, user authentication details, device metadata, location, activity or class associated with the breath-move, activity type, class type, date, time, region, user device hardware details, system details, membership level details, user points or rating, user activity history, user purchase history, user preferences, file encryption standards, music, audio, lighting conditions, a combination thereof, and the like. In some embodiments, metadata related to the context may be retrieved from user model 70, context model 75, activity model 65, exercise platform model 80, user device metadata 16 and the like based on an ID provided. In some embodiments, the user is provided with a method, such as a graphical user interface (GUI) in application 18 or voice command system in exercise platform 85 in which they may select an activity and/or provide additional context information about the session.

Receive input characterizing breath 305 comprises executable instructions that when executed by a hardware processor cause the processor to transmit control signals to one or more sensors to perform measurements related to the user's breathing. Sensors such as pressure sensors, acoustic sensors, image sensors, video sensors, humidity sensors, oximetry sensors, acceleration sensors, resistive sensors, brain sensors, multimodal sensors, and the like may be used to make such measurements. The measurements received characterize the user's breath over a duration and are used in relationship to sensor data related to the user movement to calculate a breath-move interrelation evaluation. In some embodiments, the system recognizes specific patterns and ratios of inhalation, pause, exhalation, pause, multiple inhalations in a single cycle, multiple exhalations, circle breathing with simultaneous inhalation and exhalation, combinations, and the like. In some embodiments the input is previously recorded and stored on user device 10 memory 13 or elsewhere in the system. This input is then received and evaluated by physiological analyser 40 on server 20. In some embodiments, physiological analyser 40 may be provided on user device 10.

Receive input characterizing movement or position 310 comprises executable instructions that when executed by a hardware processor cause the processor to transmit control signals to one or more sensors to perform measurements related to the user's movement and/or location. Sensors such as accelerometers, gyroscopes, Global Positioning System (GPS) sensors, camera sensors, video motion sensors, inertial sensors, Passive Infrared (PIR) sensors, active infrared sensors, Microwave (MW) sensors, area reflective sensors, lidar sensors, infrared spectrometry sensors, ultrasonic sensors, vibration sensors, echolocation sensors, proximity sensors, position sensors, inclinometer sensors, optical position sensors, laser displacement sensors, multimodal sensors, and the like may be used to make such measurements. A single sensor device may be used to measure both breath and movement. The measurements received characterize the user's movement, stillness, and/or location over a duration and are used in relationship to sensor data related to the user breath to calculate a breath-move interrelation evaluation. In some embodiments, the system may determine micro-shifts in meditative posture, for example hand, head, and neck movements. In some embodiments the input is previously recorded and stored on user device 10 memory 13 or elsewhere in the system. This input is then received and evaluated by physiological analyser 40 on server 20. In some embodiments, physiological analyser 40 may be provided on user device 10.

Map breath and movement/position 315 comprises mapping the sensor data such that the breathing pattern and the movement pattern are aligned based on timestamps, compensation for calculated sensor lag, calibration data, and the like.

Identify breath-move type 320 comprises identifying a type associated with the sensor data characterizing a breath pattern and/or movement/location and/or other context data. In some embodiments, a breath-move pattern is associated with one or more of a specific activity, a pattern of specific activities, a specific activity type, a pattern of specific activity types, an intensity of activity, an intensity of activity type, a defined set of wellness/fitness instructions, a category for a defined set of wellness/fitness instructions, a user specified intention, a user specified preference, a movement classification such as no/limited movement, isometric movement, isotonic movement, eccentric movement, concentric movement, a pattern of movement classifications, a breathing classification such as aerobic, anaerobic, meditative, a specific target respiratory rate and/or patterns, a pattern of breathing classification, a combination thereof, and the like.

Based on the breath-move type identified 320, executable instructions verify whether a type archetype defined? 325 that corresponds to the breath-move type. If yes, retrieve additional data associated with the breath-move archetype 330 will retrieve data associated with the BMIE. In some embodiments, the archetypes and data associated within them are stored in one or more of BMIE model 60, BMIE representation model 62, exercise platform model 80, activity model 65 and/or BMIE repository 68. In some embodiments, archetypes are defined within the BMIE generator 45 executable instruction logic. In some embodiments, there is a calculated interrelation and/or interrelation model which contains underlying values which may be used to generate a BMIE.

Retrieve additional data associated with the breath-move archetype 330 includes retrieving such data as a preferred breath-move interrelation evaluation associated with the BMIE type, a preferred BMIE map, a cohort BMIE and/or BMIE map, an instructor BMIE and/or BMIE map, model user, an AI and/or machine language generated, preferred BMIE and/or preferred BMIE map, and/or a combination thereof.

In some embodiments, there method can include receive additional physiological data 340 that retrieves additional physiological sensor data that can be used to refine a BMIE or be displayed/communicated in combination with a BMIE representation. Additional physiological sensor data includes such data as heart rate (HR), heart rate variance (HRV), blood pressure, brain activity, velocity, muscle activation, body temperature, oxygen levels (Sp02), CO2 levels, sweat, biomarkers, autonomic nervous system activity, and the like. In some embodiments, additional other data such as one or more of elevation, terrain, temperature, wind conditions, precipitation, tides and/or currents, and the like are received and integrated within the BMIE generation and/or representation methods.

Analyse inputs 345 identifies the data available and interrelations between the sensor, related model data, and/or contextual data which has been received. The breath pattern data is associated with the movement data. In some embodiments, additional activity-based data is used to determine whether the associations and interrelations identified in the analysis match, vary, and vary to what degree from preferred interrelations and associations between the data. In some embodiments, additional physiological data is also analysed. In some embodiments, the associations and interrelations are compared to one or more of an instructor, a preferred pattern, a preferred pattern associated with a specific skill level, a preferred pattern selected by the user, and the like. In some embodiments, interrelations are calculated using methods such as those in steps 315-345 and in some embodiments this calculation references other interrelation values and/or models.

Generate Breath-Move interrelation Evaluation (BMIE) 350 generates values based on the underlying evaluation logic. In some embodiments, the underlying evaluation logic calculates a correlation between the breath and the movement, a coherence score or code, a code indicating guidance for better coherence, a set of numeric values which may be used to generate one or more graph or wave pattern, a combination thereof, or the like.

Provide a representation of the Breath-Move Interrelation Evaluation (BMIE) 355 determines the representation to provide at a user device 10. This representation may take a number of forms such as an overlay on a video depicting the movement, a symbol or set of symbols, a numeric coherence rating, guidance on techniques to achieve a preferred interrelation between breath and movement, an inhalation/exhalation indicator which shows user variance from a preferred pattern, a movement indicator which shows user variance from a preferred pattern, an email summary, an audio indicator tone or music, an audio guidance, an alternative instructional video or adjustment to an instructional video, a “redo” option and/or tutorial, a graph or chart, an email summary, content on a summary tab in an application, and/or a combination thereof. In some embodiments, the BMIE representation is provided as a summary of a collection or series of BMIE values calculated over the duration of an activity. The determination of the BMIE representation type to provide can factor user device capacity, output device capacity, user preference, activity, activity type, user previous engagement with representation types, user movement pattern, user breath pattern, a combination thereof, and the like.

Provide representation of BMIE 355 provides the representation to the user. In some embodiments, the BMIE representation is presented near simultaneously to the user performing the activity, in some embodiments the BMIE representation is provided after the user activity or a portion of the user activity. In some embodiments, the BMIE representation is provided in response to a user action or trigger, in some embodiments by default, and in some embodiments a combination. More than one type of BMIE representation may be provided and/or to a user for a specific point in the activity duration, and/or a duration of the activity. For example, a user may select to turn on/off audio guidance during a workout and may also select whether to view a coherence rating and/or symbol showing the breath pattern.

In some embodiments, update representation of BMIE based on changing inputs 360, provides a continuous near real-time BMIE representation output to the user during the activity. In some embodiments, the updates are combined to create a summary of the BMIE during the activity that is subsequently provided to the user. In some embodiments, there is a combination of near real-time streaming feedback and summary feedback. In some embodiments, the ongoing updates to the BMIE representation update a numerical rating associated with the activity which can function as an independent coherence score and/or a factor within a general wellness, expertise, and/or membership score.

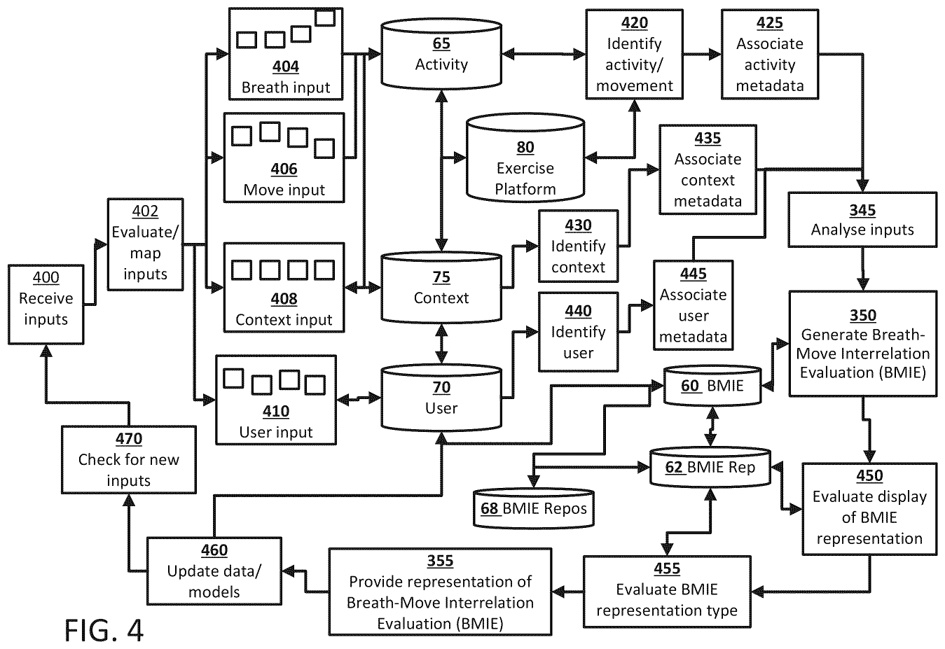

FIG. 4 shows aspects of a method for generating and/or providing BMIE based on a sensor input characterizing a breath pattern and a movement and/or location.

Input values and data models of FIG. 4 and in embodiment examples are exemplary in nature. Data elements and steps may be omitted, re-ordered, and/or added without departing from the scope of the disclosure. In some embodiments, data models activity 65, context 75, user 70, BMIE 60, BMIE representation 62, exercise platform 80 are pre-populated and updated during aspects of the method of generating and/or providing breath-move interrelation evaluations. The FIG. 4 example expands on processing operations in FIG. 3 showing additional data access, update, and exchange including data models intercommunicable and related to steps in methods to generate and provide a breath-move interrelation evaluation.

Receive inputs 400 includes receiving recorded and streamed input or a combination. Streamed input comprises real time and near real time: sensor data, streamed video and/or images, audio recording data, augment reality data, virtual reality data, mixed reality data and/or a combination. Recorded input comprises sensor data, streamed video and/or images, augment reality data, virtual reality data, mixed reality data, and/or a combination. Such inputs can include and be augment by user, application, and/or system inputs which provide additional related data such as a context, user ID, user type, user membership, activity, activity type, exercise activity ID, exercise platform ID, and the like. In some embodiments, recorded and streamed inputs are processed separately, and different analyses are applied based on the whether the input is a previously recorded or real time/near real time.

In one embodiment, a combination of recorded data and streamed input are combined based on a rebroadcast on-demand event with livestreaming participants. In one embodiment, the rebroadcast on-demand event is a class, fitness activity, concert, training session, private workout, or the like. In some embodiments, multiple user streams are simultaneously evaluated for methods related to this process and generate breath-move interrelation evaluation (BMIE) 350 and evaluate display of BMIE representation 450 and evaluate BMIE representation type 455 evaluate both livestreaming and recorded inputs associated with sensor input related to breath input 404 move input 406, context input 408, and/or user input 410. In some embodiments, a user's BMIE is evaluated in relationship to an instructor and/or other user's BMIE.

Evaluate/map inputs 402 identifies specific data related to breath input 404, move input 406, context input 408, and user input 410 and associates the data with appropriate model/repository activity 65, exercise platform 80, context 75, user 70, BMIE 60, BMIE representation 62 and/or BMIE repository 68.

Identify activity/movement 420 identifies the activity, activity type, series of activities, series of activity types associated with the sensor data. In some embodiments, exercise platform 80 provides activity metadata associated with a fitness class, fitness activity, dance class, wellness class, and/or wellness activity. In some embodiments the user selects an activity. In some embodiments, the activity is associated with an identified location and/or time. Associate activity metadata 425 associates additional data available in the system.

Identify context 430 identifies such factors as a user, a user ID token, session ID, hardware capacities, software capacities, regions, encoding types, lighting, camera resolution, timestamps, exercise class context, workout context, membership level, user role, system hardware and other metadata associated with the input 400. In some embodiments, the input context identifies one or more of whether the input is live or recorded, the time of the recording, the duration of the recording, the qualities of the activity depicted in the recording, and whether the user depicted is an instructor, educator, or influencer. In some embodiments, the context includes previously generated BMIE values and/or BMIE representations associated with the user, current activity, previous activity, current activity type, and/or a combination. Associate context metadata 435 associates additional data available in the system.

Identify user 440 identifies one or more of the user depicted in input 400. The user model 70 may be updated with information related to the input received associated with the user and/or BMIEs related to the user. In some embodiments user data related to user activity history, user preferences, user devices, user BMIE history, user type, user preferences, user membership, user purchase history, user wellness history, and the like are associated with the user. Associate user metadata 445 associates additional data available in the system with the user input.

In some embodiments, one or more baseline BMIE is generated for a user. This baseline may be associated with a user state or characteristic such as being at rest, associated with a specific heart rate (HR), HR range, activity, activity type, activity pace, activity duration, or the like. The baseline BM IE may be related to cyclical movement (e.g. running, walking, swimming, dancing). The baseline BMIE may be aligned with the cyclical movement, such as strides or strokes of the cyclical movement. This baseline BMIE may be regenerated based on time, changes in user fitness and/or activity levels, changes to the cyclical movement or the like. In some embodiments, a baseline BMIE is generated based on the a user's resting BM IE (or a user's BM IE associated with a specific activity) and machine learning or AI models can be used to generate and refine baseline BM IEs associated with the user and other activities.

Analyse inputs 345 analyses the inputs received including specific data related to breath input 404, move input 406, context input 408, and user input 410 and other related metadata 425 435 445. The inputs are factors in the breath-move interrelation that is calculated. In some embodiments there is a calculated interrelation and/or interrelation model which contains underlying values which may be used to generate a BMIE. In some embodiments, the association between breath-move is evaluated based on models of preferred breath-move relationships based on activity, activity intensity, activity type, wellness recommendation, intended wellness outcome, a model of preferred breath-move interrelations, a skill level based model of preferred breath-move interrelations, a training-based model of preferred breath-move interrelations, a community-based model of preferred breath-move interrelations, a machine learning and/or AI based model of preferred breath-move interrelations and/or a combination of such preferred interrelation types.

Generate BMIE 350 generates a BMIE associated with the input breath pattern and movement and/or location. See FIG. 3, 5-9 for additional methods and aspects associated with generating, regenerating, and customizing a BMIE and/or BMIE representation. In some embodiments, the generated BMIE is compared to a baseline BMIE associated with the user. In some embodiments, a BMIE representation is only provided to a user when the BMIE that is generated varies from a baseline BMIE within threshold determined by a specific factor or according to a specific formula, for example. In some embodiments, a BMIE representation includes an indication of how the BMIE relates to one or more baseline BMIE. The baseline BMIE may be a preferred BMIE or a preferred BMIE type.

A baseline BMIE, a characteristic associated with a baseline BMIE, breath characteristic associated with a user's baseline BMIE, a movement characteristic associated with a user's baseline BMIE, a physiological characteristic associated with a user baseline BMIE, may provide one or more thresholds for one or more of receiving and/or evaluating data, generating a BMIE, generating a BMIE representation, providing a BMIE representation, determining the type of BMIE representation to provide, or the like. The baseline BMIE can be use to compare the generated BMIE to ensure it is within the one or more thresholds prior to providing the generated BMIE.

Evaluate display of BMIE representation 450 evaluates such factors as the current context, key BMIE factors, the number/type of BMIE that might be effectively displayed. In some embodiments, the evaluation is informed by machine learning in the BMIE model. Factors, for example, such as user history, preferences, previous engagement with BMIEs may be used to evaluate display of a BMIE indication, and/or BMIE representation for evaluate display of BMIE representation 450 and evaluate BMIE representation type 455 including prioritization, location in the interface, means and style of providing the representation.

Evaluate BMIE representation type to provide 455, evaluates the BMIE representation, and possible alternative BMIE representations, for the breath-move sensor input based on the context. In some embodiments, a user can select the type of BMIE representation they are provided. In some embodiments, provide representation of breath-move interrelation evaluation (BMIE) 355 triggers a delayed process that for example may be provided when a user completes a workout, game, or other activity. In some embodiments, the BMIE representation is provided in a different context within the system, for example the user may be engaged in a swimming workout activity and provide representation of breath-move interrelation evaluation (BMIE) 355 may email a BMIE performance summary to the user for later review after the workout. In some embodiments, the user BMIE and/or a representation of the user BMIE may be provided to a second user, for example a coach or instruction, and the second user may, based on the BMIE representation, provide the user with guidance. In some embodiments, the BMIE representation model 62 is updated based on the BMIE provided, engagement with the BMIE, performance improvements associated with the BMIE, endurance improvements associated with the BMIE, improved wellness outcomes associated with the BMIE, increased engagement with the application associated with the BMIE, improved breath-move coherence, purchases resulting from the BMIE provided and the like.

In various embodiments, the method in FIG. 4 may make use of machine learning types based on one or more of a combination of unsupervised, supervised, regression, classification, clustering, dimensionality reduction, ensemble methods, neural nets and deep learning, transfer learning, natural language processing, word embeddings, and reinforcement learning. Such machine learning may be performed using processes and evaluation tools such as K-Means Clustering, Hierarchical Clustering, Anomaly Detection, Principal Component Analysis, APriori Algorithm, Naïve Bayes Classifier, Decision Tree, Logistic Regression, Linear Regression, Regression Tree, K-Nearest Neighbour, AdaBoost, Markov Decision Processes, Linear Bellman Completeness, Policy Gradient, Asynchronous Advantage Actor-Critic (AC3), Trust Region Policy Optimization (TRPO), Proximal Policy Optimization (PPO), Reinforcement Learning from Human Feedback (RLHF), Generative Adversarial Network (GAN), Recurrent Neural network (RNN), Convolutional Neural network (CNN), Deep Q Neural Network (DQN), C51, Distributional Reinforcement Learning with Quantile Regressions (QR-DQN), Hindsight Experience Replay (HER) and the like. In one embodiment, the machine learning is based on one or more of user feedback, user engagement, user purchases, user BMIE engagement, user BMIE feedback, user BMIE activity engagement, purchases resulting from a BMIE, BMIE type feedback, BMIE representation type engagement, activity participation resulting from a BMIE representation.

In some embodiments, there are archetypes which store breath-move interrelation logic associated with specific activity, specific heart rates, breath rates, specific activity patterns, categories of activity, categories of heart rate, categories of breath rates, categories of activity patterns, specific users, specific categories of users, and the like. These archetypes include a logic and/or preferred interrelation between breath and movement. In some embodiments, there are templates of BMIE patterns associated with an archetype. These templates can define a map or series of breath-move interrelations that constitute a preferred pattern of interrelations over a duration while engaged in one or more activities. Within the map (or template) individual points are associated with breath-move interrelation evaluations which determine the interrelation within a point of time or micro-duration. From the map and/or specific BMIEs for a point in time a representation of the BMIE can be generated which gives the user insight into the BMIE associated with the BMIE for a point in time or micro-duration and/or the BMIEs associated with a longer duration or map.

Update data/models 460 updates one or more of the data models including the user metadata, with values associated with the BMIE values, BMIE representation, user engagement with BMIE representations, and the like.

Check for new inputs 470 is indicative of the ongoing receiving of sensor and other data. Sensor data frequency example ranges include once per second, 1 hz sample rate (or lower) to 20,000 times per second 20 Khz (or higher) such as the sample rates found in accelerometers. When receiving inputs where the context input 408 and user input 410 have not changed, identification processes such as 430, 440 and association processes such as 435, 445 may be omitted in some embodiments.

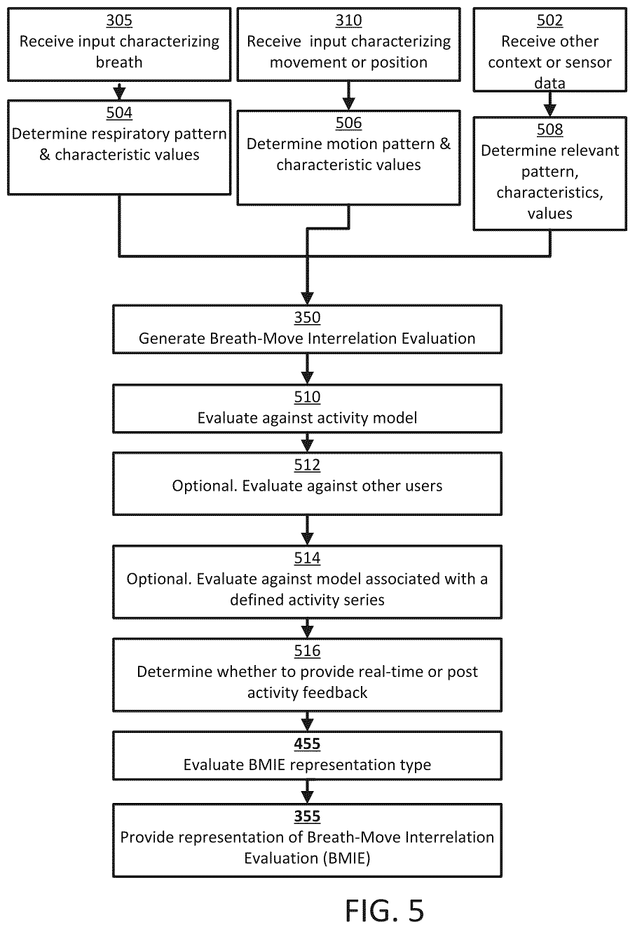

FIG. 5 shows an example method associated with generating BMIE and/or providing a BMIE representation. The method receives input characterizing breath 305, input characterizing movement or position 310, and other context or sensor data 502. This data can be streamed, recorded during a previous activity session, and/or a combination. Based on the breath inputs the system determines respiratory pattern and characteristic values 504 can include such factors as depth, input through mouth and/or nose, depth of inhalation based on throat, chest, belly expansion and/or tension, consistency, oxygenation, velocity, rate, volume, coherence, and the like.

Determine motion pattern and characteristic values 506 can include factors such as velocity, linear motion, rotary motion, reciprocating motion, oscillating motion, angle of velocity, orientation, angle of rotation, vibration, GPS location, uniformity of motion, rate, and the like.

Determine relevant pattern, characteristics, values 508 can include such factors as contextual data entry or selection, user ID, hardware ID, instructional session ID, system ID, heart rate, heart rate variability, muscle fatigue measures, pressure, brain activity, velocity, muscle activation, body temperature, oxygen levels (Sp02), sweat, biomarkers, current and or previous wellness rating, current and/or previous BMIE coherence rating, user physical environmental context, other contextual and physiological factors.

In some embodiments, there are provided systems, methods, and executable instructions for synchronizing sensor input including the one or more input characterizing a user breathing pattern and one or more input characterizing a user movement. This synchronization may include a means of user calibration, date-time stamp verification and alignment, establishing master-slave sensor relationships, using a timing transport protocol such as RIG (Inter-Range Instrumentation Group), GPS PPs (Global Positioning System Pulse Per Second), NTP (Network Time Protocol), EtherCAT (Ethernet for Control Automation Technology) PTP V2 (Precision Time Protocol) and the like to ensure sensor synchronization.

Generate Breath-Move Interrelation Evaluation 350 generates a BMIE based on the interrelation of the breath pattern and movement in a specific moment or micro-duration. The calculation may factor other characteristics related to the inputs received.

Evaluate against activity model 510 evaluates the generated BMIE against the expected BMIE associated with an activity model. The degree of variance in breath pattern, motion pattern, and/or the interrelation thereof is calculated. The generated BMIE may be augmented with additional data related to its evaluation within the context of an activity model.

In some embodiments, evaluate against other users 512 evaluates the BMIE based on the BMIE of other users. These other users can be instructors, cohorts, friends, members of a group, educators, representative users with similar attributes, user models generated based on machine learning and/or AI, and the like.

In some embodiments, evaluate against model associated with a defined activity series 514 includes defined activity series such as a series of yoga poses, a set of exercises, a series of dance movements, a dance routine, a warmup series, cooldown series, a cycling pace, a cycling technique, a series of cycling paces/techniques, a running pace, a running technique, sets of running paces/techniques, a swimming stroke, sets of swimming strokes/paces, a class with a predetermined format, a class which has been mapped to a format, and the like.

Determine whether to provide real-time or post activity feedback 516 may evaluate factors such as device type, activity type, activity intensity, user preference, a combination, and the like. In some embodiments, by default a summary BMIE map is provided post-activity and/or stored for comparison with other summary BMIE maps. In some embodiments, real-time guidance to achieve a preferred BMIE is provided to the user during the activity. In some embodiments, a BMIE symbol is provided as an overlay on the user's reflection as they perform the activity. In some embodiments, the BMIE is calculated as a factor within a wellness rating and/or overall BMIE coherence rating.

Evaluate BMIE representation type 455 determines the representation type based on factors such as whether the representation is presented real time, near real time, or subsequent to the activity, device type, activity type, activity intensity, user preference, and the like, or a combination. In some embodiments, more than one BMIE representation type is determined. In some embodiments, a BMIE map is provided.

Provide representation of Breath-Move Interrelation Evaluation (BMIE) provides the representation such that it is displayed and/or provided through user device 10 and/or output 17. In some embodiments, more than one BMIE representation is provided.

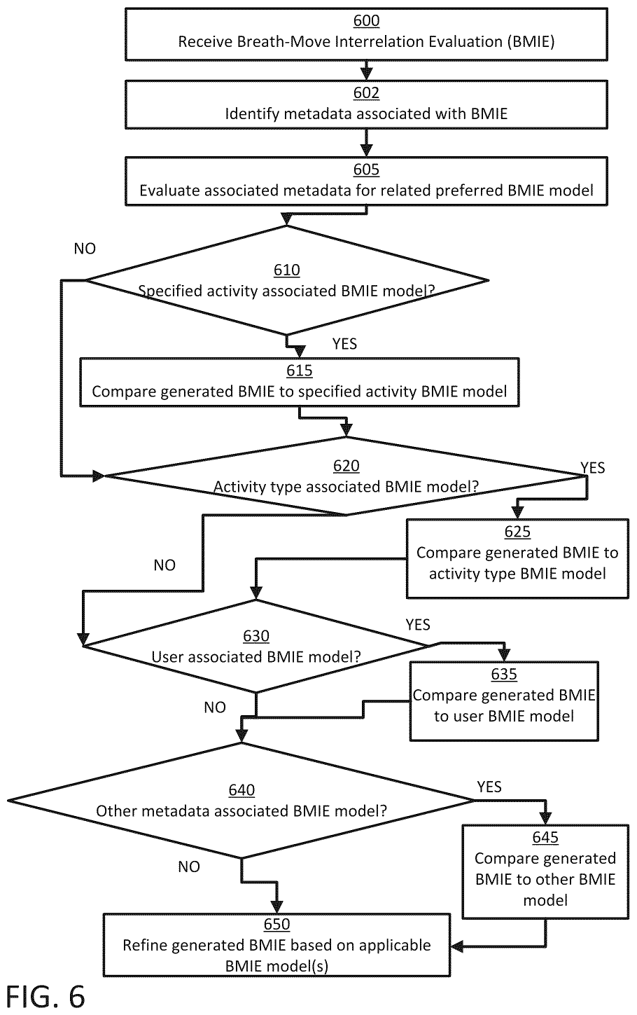

FIG. 6 shows an aspect of a method associated with generating BMIE in accordance with an embodiment.

Receive Breath-Move Interrelation Evaluation (BMIE) 600 receives a BMIE that has been previously generated based on inputs associated with a breath pattern, motion and/or location, and/or other data.

Identify metadata associated with BMIE 602 identifies one or more factors associated with the BMIE. Associated metadata can include metadata such as the user, user type, user membership, user history, user groups associated with a user, activity associated with user, activity type associated with user, user skill level, activity, activity types, activity intensity, activity history, training program associated with activity, fitness class associated with activity, wellness class associated with activity, combinations thereof and the like. In some embodiments, one or more of a previously generated BM IE that are associated with the BMIE, previously identified models that are associated with the BMIE, previously identified maps that are associated with the BMIE, previously identified archetypes that are associated with the BM IE are identified.

Evaluate associated metadata for related preferred BMIE model 605 evaluates the identified metadata to identify preferred BMIE models. Models may contain archetypes of BMIE preferred patterns based on general breath-movement interrelation logic, maps of BMIE patterns associated with a specific activity, set of activities, or activity type. In some embodiments, instructor led activities (real-time, recorded, virtual, and the like) are associated with a BMIE map. In some embodiments, specific types of training related to a specific activity are associated BMIE patterns defined in a map. In some embodiments, a series of actions for example a set of yoga poses, stretches, weightlifting activities, are associated with a defined BMIE pattern map. In some embodiments, BMIE maps are defined and/or customized based on machine learning models, by activity experts, by users, by capturing a series of BMIEs associated with a selected individual performing an activity or set of activities, by capturing a series of BMIEs associated with an individual with one or more specific characteristic such as skill level, age, region, training method, community, gender, performing an activity or set of activities, by capturing a series of BMIEs associated with a cohort of individuals performing an activity or set of activities, by capturing a series of BMIEs associated with a cohort of individuals with one or more specific characteristic such as skill level, age, region, training method, community, gender, performing an activity or set of activities.

Specified activity associated with BMIE model? 610 determines whether the activity is associated with one or more BMIE model, archetype, map or the like. In some embodiments, specific classes and/or group activities are associated with a map. This map may be predetermined, associated with the BMIE of an instructor, educator, or expert performing the activity, generated based on encoded activity metadata within a representation of the activity such as a defined workout, or a combination. In some embodiments an activity type is combined with one or more activity characteristics to determine associated models. For example, a map may be associated with cycling at a specific pace, a specific terrain type (actual or simulated), a specific user heartrate, a specific user training model, a specific user training goal (cardio, strength building, endurance, relaxation, stimulation, and the like), or similar and/or a combination thereof.

If Yes, the specified activity is associated with one or more BMIE model, Compare generated BMIE to specified activity BMIE model 615 is performed to determine coherence and/or variance between the BMIE received and the BMIE associated with the model. In some embodiments, when a closely matching BMIE model is found based on the metadata, further checks are not performed. In some embodiments, all potential matching BMIE models are identified by the method and are used in combination to refine the generated BMIE based on applicable BMIE model(s) 550.

If No, the specified activity is not associated with a BMIE model, the Activity type associated with BMIE model? 620 determines whether there is a BMIE associated with the activity type. In some embodiments the activity type is hierarchical and covers a broad category of activities with more specific models for subcategories identified within the broadest category.

If Yes, Compare generated BMIE to activity type BMIE model 625 compares the BMIE received to one or more activity type BMIE model. For the purposes of example, a hierarchy of activity types models might be something like the following: activity involving motion, cardiovascular activity, full body, swimming, front crawl stroke, or the like. Depending on the BMIE archetypes and maps associated with the model, maps may be available at different levels within the hierarchy of activity type. In some embodiments, the map associated with the most specific applicable level of the hierarchy is applied. In some embodiments, after the activity type comparison, the method continues to determine BM IE models associated with the user.

If no, the activity type is not associated with one or more BMIE model, then User associated with BMIE model? 630 checks whether there is one or more BMIE model associated with the user. BMIE models associated with the user may include models selected based on user preference, user activity history, user BMIE history, individuals the user follows, instructors, coaches, educators and the like with whom the user is or has previously engaged, user purchase history, activity associated with user purchases history, a combination, or the like.

Compare generated BMIE to one or more user associated BMIE model 635 evaluates the current BMIE in relationship to these other user associated BMIE models.

No/then Other metadata associated with BMIE model? 640 evaluates whether other contextual metadata associated with the BMIE (Identify metadata associated with BMIE 602) is associated with one or more other models. In some embodiments, there are one or more general model which may be applicable based on metadata.

If yes, Compare generated BMIE to other BMIE model 645 compares the generated BMIE to one or more other models.

Refine generated BMIE based on applicable BMIE model(s) 650 refines the BMIE provided to the user based on one or more BMIE models identified in the process. In some embodiments, one or more of multiple BMIEs are calculated, composite BMIEs that compare the user's BMIE to multiple BMIE models, collections and hierarchies of BMIEs, are generated. In some embodiments, if no applicable model is identified, a BM IE representation of the user's breath-move interrelation evaluation is provided that provides details of the interrelationship without comparison to a preferred BMIE.

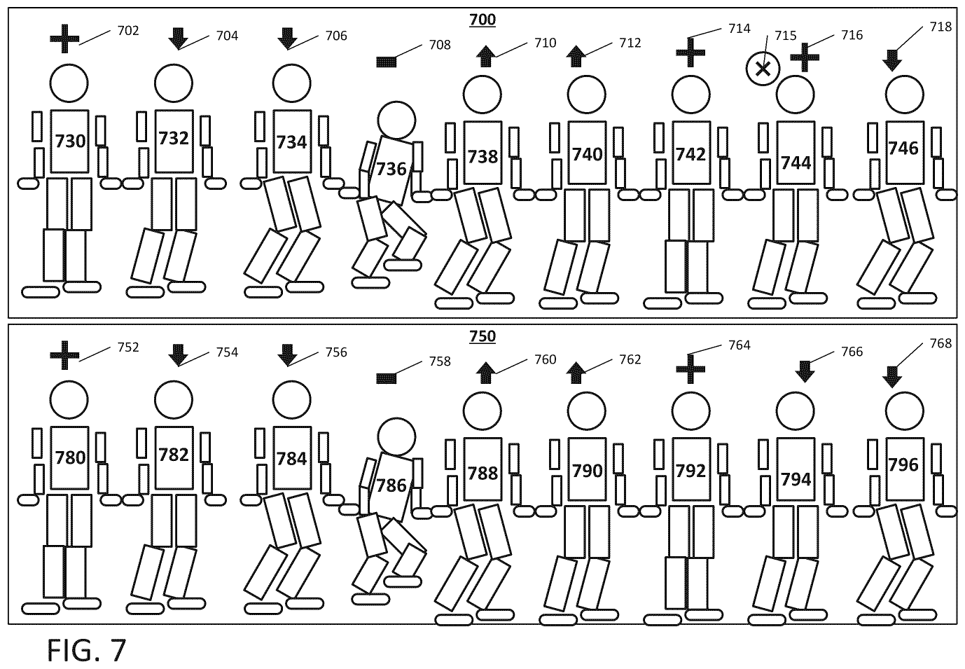

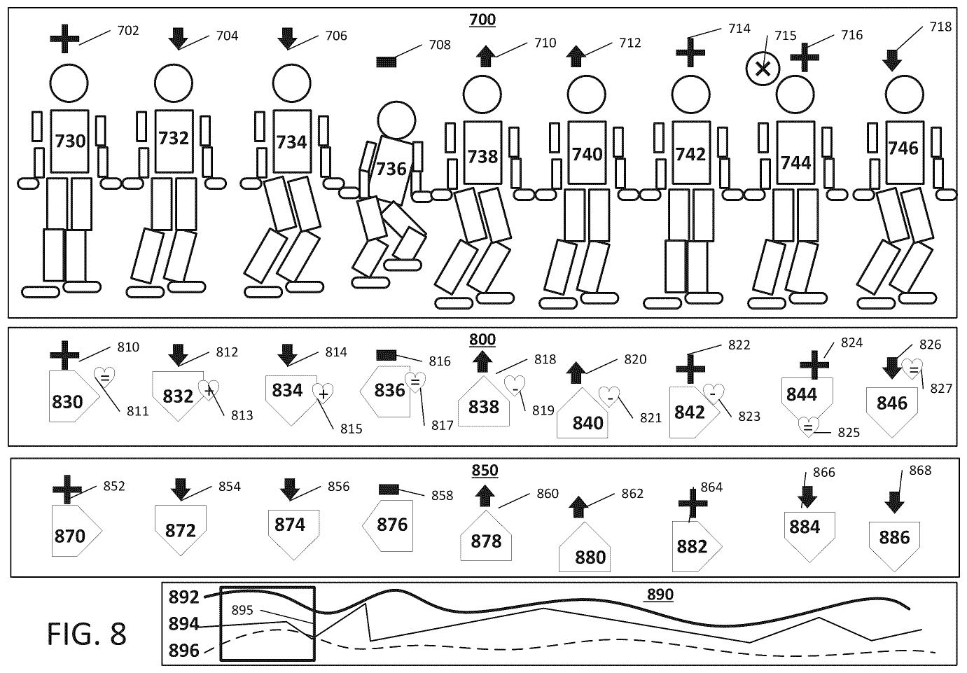

Turning to FIG. 7, an example related to breath-move interrelation evaluation is provided. FIGS. 7-8 represents a simplification for the purposes of discussion. Sensor data and the evaluation of sensor data typically may be captured/performed at a significantly greater frequency, with greater gradations of data values, and additional data aspects than the figures suggest.

In this example, correspondence between the breath-move interrelation evaluation of user 700 engaged in squat type motion and an exemplary model 750 (instructor, hero, activity model, preferred model, skill-based model, training method based model, machine learning model, embedded activity map, combination or the like) is shown.

In one known preferred breath-move pattern, the inhalation occurs just prior to an eccentric (muscle-lengthening) portion of the motion, and the exhalation occurs during a concentric (muscle-shortening) portion of the motion. In some embodiments, a generalized model to evaluate the breath-move interrelation evaluation against the user's eccentric and concentric motion is provided. In some preferred breath-move patterns, there is also preference for inhalation through the nose and exhalation through the mouth. Some embodiments include evaluating whether breathing is performed through the nose and/or mouth and including this data in the BMIE and in some representations and/or representation types. Additional characteristics such as the depth and/or velocity of the breath may also be factors.

In FIG. 7, user 700 sensor data related to a breathing pattern is represented with indicators 702, 704, 706, 708, 710, 712, 714, 716, 718, where 702, 714, 716, represent a pause when the user is neither inhaling or exhaling post exhalation, 708 represents a pause when the user is neither inhaling nor exhaling post inhalation, 704, 706, 718, represent inhalation, 710, 712, represent exhalation. Similarly, user motion is captured using a sensor. In this example, user movement captures 730, 742 represent the user in a paused neutral stance where the user is not actively moving and 736 represents a pause when the user is in a squat position, eccentric motion is indicated in movement capture data points 732, 734, 744, 746 and concentric motion is indicated in movement capture data points 738, 740.

In this example, there is one point 715 identified where user 700 breath-move interrelation evaluation does not correspond to model 750. At this point, given eccentric movement 744 breath holding 716 is not recommended in the preferred model at movement 794 and there is inhalation 766. In some embodiments, a breath-move interrelation evaluation representation for user 700 would provide an indication of this lack of coherence to the preferred model 750. This indication might include for example one or more of a warning, guidance, or instruction for the user, changing the color or another visual aspect of a breath indicator, changing the color or another visual aspect of a breath-move interrelation indicator, changing a rating, adding, removing, or changing a visual element, adding, removing, or changing an audible element, adding, removing, or changing a tactile element, adding, removing, or changing a lighting element, adding, removing, or changing music, adding, removing, or changing a video, adding, removing, or changing an avatar, adding, removing, or changing an animation, adding, removing, or changing a badge. These same types of means of indication may be used to indicate coherence, one or more range related to coherence or lack of coherence, the specific aspect of breath or movement which is coherent, within one or more range related to coherence, or out of coherence, other biometric values associated with the BMIE.

Referring to FIG. 8, we see user 700 series of breath movement as depicted in FIG. 7. Series 800 represents sensor data associated with series 700 where movements/pauses 730, 732, 734, 736, 738, 740, 742, 744, 746 have been symbolically abstracted in 830, 832, 834, 836, 838, 840, 842, 844, 846. A number of different sensor means may be used individually or combination to determine use movement. In some embodiments sensor data 830, 832, 834, 836, 838, 840, 842, 844, 846 is associated with sensor data that indicates one or more of muscle extensions and contractions, change in physical position, specific movements in portions of the body, specific movements in specific limbs, distance traversed, terrain traversed, engagement with physical resistance, engagement with physical resistance associated with specific resistance value(s), movement of weight, movement of weight associated with specific weight value(s), displacement, distance, velocity, acceleration, speed, movement within one or more predetermined set of movement patterns, cadence, center of gravity, center of pressure, movement duration, movement phase, elevation traversed, repetitions completed, movement patterns completed, quality of motion, jerk, vibration, projectile, consistency, oscillation, elasticity, snap, combinations thereof, and the like. In series 800, motion sensor data is augmented with additional biometric data, shown as heart rate variability (HRV) in this example, with sensor value indicators 811, 813, 815, 817, 819, 821, 823, 829, 827. In some embodiments, the BMIE includes biometric factors such as body temperature, sweat characteristics, heart rate, heart rate variability, blood glucose, blood pressure, oximetry, brain activity, EEG, and/or other biomarkers.

In FIG. 8, exemplary model 750 of FIG. 7 is depicted in abstracted model 850 where symbolic data values such as numbers, ratings, characteristic labels, scores, ranges, and the like are applied to the preferred model. In some embodiments, additional factors other than the breath pattern represented by data points 852, 854, 856, 858, 860, 862, 864, 866, 868, and movement pattern represented by data points 870, 872, 874, 876, 878, 880, 882, 884, 886, are represented and/or evaluated in preferred model 850.

Referring to series 890, we see sensor data associated with user 700 motion pattern where graph 892 represents the user breath pattern, graph 894 represents one or more additional biometric or other sensor inputs, and graph 896 represents user movement pattern. In series 890, we see segment 895 within the rectangular focus segment where this graph segment indicated by 895 corresponds with user series 700 where the single squat is performed. In some embodiments, sensor data associated with a user breath-move interrelation evaluation is graphed and evaluated against graphs associated with one or more preferred breath-move interrelation evaluation. In some embodiments, the BMIE is a graph or map of data values.

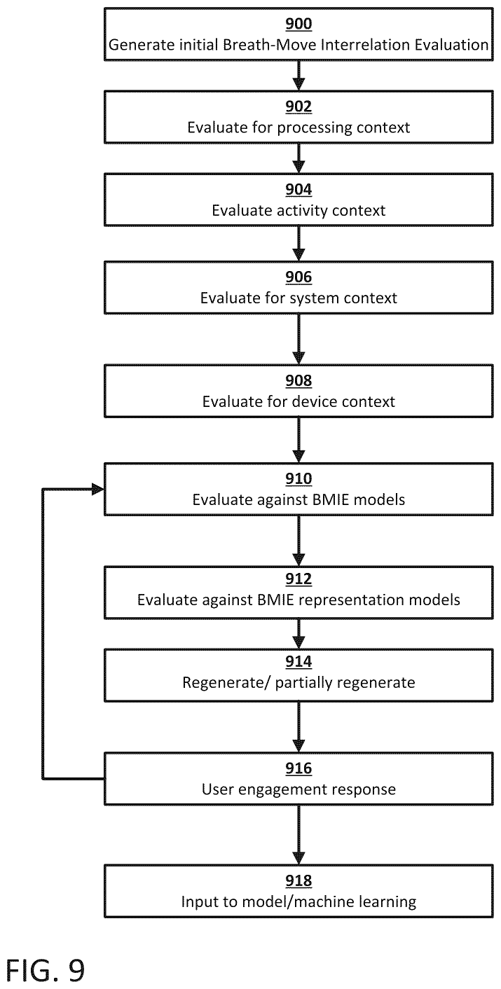

Turning to FIG. 9, an example method associated with generating BMIE and/or providing a BMIE representation is shown. Generate Breath-Move Interrelation Evaluation 900 may include generating such breath-move interrelation evaluations as are generated through methods shown in FIG. 3-6. In some embodiments, an initial BMIE is generated and then further refined, augmented, reduced and/or replaced as further evaluation occurs. More than one BMIE may result from an evaluation process and a BMIE may be associated with one or more representations. Evaluate for processing context 902 evaluates for such factors as whether the BMIE is being generated real-time, near real-time, post activity, number of sensor data sources, number of types of sensor data, compression of sensor data, processing requirements associated with receiving and/or analysing sensor data, other data sources provided, other video processing, other data processing requirements, hardware capacity, processing system capacity, network capacity, combinations of such factors, and the like. In some embodiments, a second BMIE with more expensive processing is generated post-activity.

Evaluate activity context 904 evaluates factors such as physical location, features of physical location such as water, water depth, surface, terrain, temperature, atmospheric conditions, which user devices may be available, user privacy, shared activity contexts with multiple users performing an activity together, user safety requirement for the activity, user collaboration and/or competition in the activity, combinations, and the like.

Evaluate system context 906 evaluates factors such as multiple user devices and contexts in which BMIEs and BMIE representations may be stored, evaluated, and/or provided in the system. In some embodiments, these factors include preferences extrapolated from user engagement with the system, preferences specified by the user, machine learning about user preferences and/or system context optimisation. For example, a system may store BMIEs in a repository, a cloud server or user device depending on factors such as availability of storage options, file size, network capacity, requirements associated with generating a representation of the BMIE and/or combinations. Similarly, BMIE representations may be provided through one or more of a web application, summary emails, activity tracker applications, connected devices guidance, chat functionality, audio application, integrated components within a system. In some embodiments, BMIEs and/or BMIE representations are inputs and/or outputs to a larger social network, wellness recommendation, exercise platform, retail experience, community membership, and/or combination thereof type system.

Evaluate device context 908 evaluates factors such as for example physical location of device, physical location of user, physical location of user with regard to device, connectivity of device with a system of network, device capacities, and the like. In some embodiments, some representations require one or more specific device capacity such as a memory, processor capacity, storage, display, virtual reality capacity, augmented reality capacity, mixed reality capacity, audio output, audio input, vibration output, video output, video input, camera input, specific display qualities, heating/cooling, lighting control, and the like. In some embodiments, one or more primary and secondary device within the system are identified and evaluated to determine where to generate, store, or provide one or BMIE and/or BMIE representation.

Evaluate against BMIE models 910, evaluates the initial breath-move interrelation evaluation and one or more of the evaluated processing context, activity context, system context, device context against BMIE models. For example, these models may be generalized models of the correspondence between breath and movement, activity specific models, user specific models, community models, training method specific models, skill level specific levels, activity intention specific models, defined models associated with a predefined activity or set of activities, and the like. In some embodiments, one or more of these types of BMIE model are generated based on machine learning.

Evaluate against BMIE representation models 912, evaluates the initial breath-move interrelation evaluation and one or more of the evaluated processing context, activity context, system context, device context, BMIE models associated in relationship to BMIE representation models. For example, some BMIE representations require specific breath-move interrelation evaluation values and factors such as additional biometric data, a specific type of activity/movement associated with the BMIE, a specific activity intention associated with the activity and the like in order to be applicable, available and/or preferred. In some embodiments, one or more of these types of BMIE representation model are generated based on machine learning.

Regenerate/partially regenerate 914 may result in a regenerated, or partially regenerated BMIE or BMIE representation. In some embodiments, a BMIE representation may be augmented with an element that personalizes the BMIE or its representation. In some embodiments, additional target BMIE models are added to the BMIE representation. In some embodiments, regenerating/partially regenerating only occurs if there has been a change in one of the contexts evaluated.

User engagement response 916 includes the ongoing monitoring and evaluation of user engagement with BMIE and BMIE representations which includes for example user selection, explicit and implicit feedback, eye tracking, opening an application, email, or message, user shares, user likes, and similar forms of engagement. In some embodiments, user engagement includes engagement with the first user's BMIE or BMIE representation by a second user where the first user may be a fellow participant, instructor, educator, hero, friend, community member or the like of the second user.

Input to model/machine learning 918 includes augmenting the BMIE, BMIE representation and/or other models with data and extrapolated data generated through evaluation processes. In some embodiments, this method provides data to train one or more machine learning generated model.

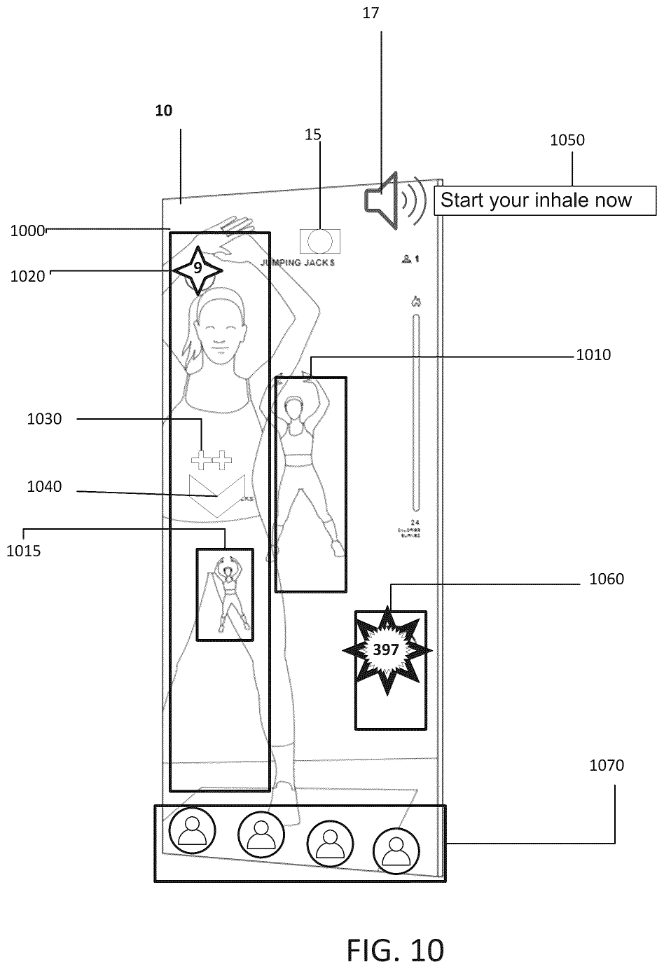

FIGS. 10-13 illustrate examples of aspects of embodiments for generating and providing breath-move interrelation evaluations and breath-move interrelation evaluation representations and example user interfaces.

In some embodiments an interface (e.g., application 15 of user device 10, web app 38 of server 20) provides a breath-move interrelation evaluation representation, in some embodiments the breath-move interrelation evaluation representation is provided through a connected device, or smart device, in some embodiments it is provided through email, system notifications, SMS (Short Message Service) message, and/or MMS (Multimedia Messaging Service) message, and in some embodiments, it is provided through a combination thereof. In some embodiments, the breath-move interrelation evaluation representation is provided within the context of an exercise environment, exercise class, meditation class, guided workout, guided meditation, or the like by adjusting the profile images, streams, screen position, audio, providing additional color, symbol, video and/or other indicators. In some embodiments, a provided BMIE and an indicator of the availability of one or more BMIE are displayed on the same user device and in some embodiments they are not. In some embodiments, the generation of a BMIE is an output or input to a system for wellness recommendations.

In some embodiments, BMIE representations may provide an indication of current movement pattern, breath pattern, and/or interrelation of breath-move pattern, an indication of preferred movement pattern, breath pattern, and/or interrelation of breath-move pattern, guidance related to current movement pattern, breath pattern, and/or interrelation of breath-move pattern, guidance related to achieving the preferred movement pattern, breath pattern, and/or interrelation of breath-move pattern and the like, summary information about the user movement pattern, breath pattern, and/or interrelation of breath-move pattern, summary information about variance between the user and one or more preferred movement pattern, breath pattern, and/or interrelation of breath-move pattern, and combinations.

In some embodiments, a user may prior, during, or post activity use an application to trigger the control of sensors and evaluation of sensor data to generate a BMIE. In some embodiments, this may include the user specifying an activity, intended activity outcome, or preferred training model. In some embodiments, participation in an in-person class, retail experience, or event, may be a factor in generating a BMIE.

In one embodiment, the BMIE representation is provided as human-readable instructions and/or guidance applicable to a real-world 3D context. In one embodiment, the instructions are provided to a coach, educator, retail assistant, leader, teacher, performer, or instructor in order for that individual to communicate the instructions to another individual in-person and/or through a live voice and/or video chat.

The BMIE may be provided by one or more of a web application, an application installed on a user device, a smart mirror device, a connected audio/music system, a connected exercise mat, a virtual reality system application, a virtual reality headset, a augmented reality system application, an augmented reality headset, a metaverse headset, a haptic glove, a game controller, a haptic garment, a retail application, a coaching application, a fitness class or studio application, a meditation application, a retail application, an email system application, a text message system application, a chat system application, a notification system application. BMIE representations may be provided in a “real-life” 3D reality environment, augmented reality environment, simulated reality environment, virtual reality environment, a game environment, a metaverse environment.

In some embodiments, the breath-move interrelation evaluation system is integrated within a retail system or social media platform. In some embodiments, an application is provided to the user to calibrate sensors, select preferences, associate one or more BMIE with a user profile, and/or indicate the activity associated with measurements. In some embodiments, this is integrated within an activity within an exercise platform or wellness system.

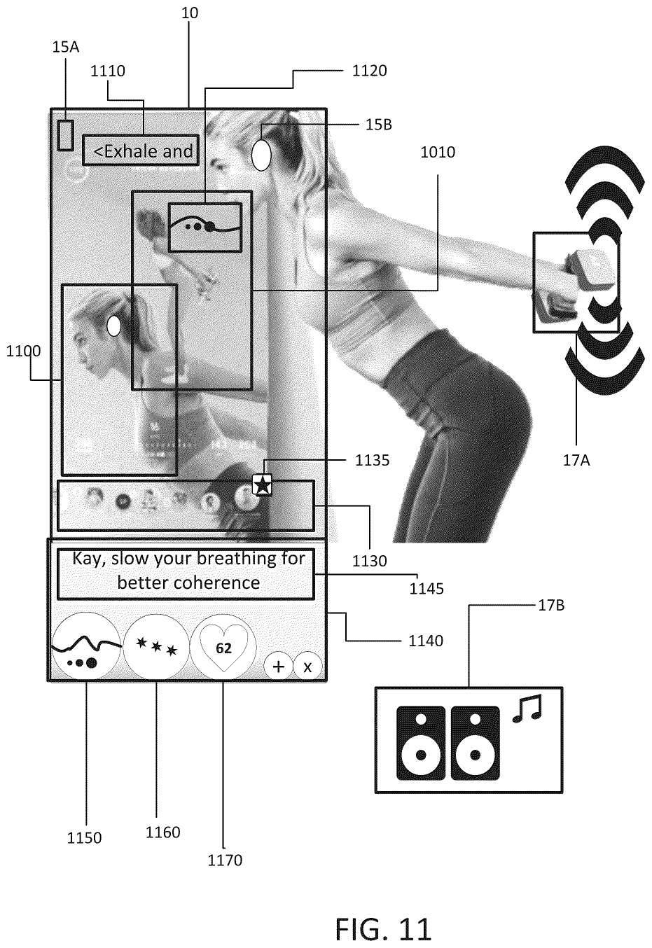

FIG. 10 shows an example embodiment in which, based on the breath-move interrelation in the BMIE, the system provides guidance to the user to assist the user in shifting their breath and/or motion to more closely align to a preferred BMIE. In this example, user device 10 is a smart mirror with input 15 camera, microphone, and/or other sensors and output 17 through an audio speaker and the smart mirror display screen. In this example we see a reflection of the user 1000 where the reflection has been augmented by elements such as coherence rating 1020, adjustment guidance 1030, and breath pattern indicator 1040 as well as instructor video 1010 and overall points 1060 which in some embodiments coherence rating 1020 may be a factor. In some embodiments, (as shown in FIG. 11) additional graphs and physiological data may be provided. In some embodiments, the user may see BMIE representations associated with instructor 1010 or other users such as 1015 or 1070.

In addition to visual BMIE representation feedback, in this example audio BMIE representation feedback 1050 is provided. In some embodiments, BMIE representations may provide an indication of current movement pattern, breath pattern, and/or interrelation of breath-move pattern combined with a guidance toward a preferred movement pattern, breath pattern, and/or interrelation of breath-move pattern which may include such factors as summary, correction, reinforcing positive feedback, and/or other forms of feedback.

The preferred BMIE may be based on a number of different factors including one or more of an instructor 1010 BMIE, a BMIE map encoded within the set of activities provided, the BMIE of other participants 1015 1070, or another preferred BMIE model, archetype, or map. In some embodiments, the guidance is provided through audio indications such as voice instruction, an indicator tone, music, a change in music, and like. BMIE representations and guidance based on BMIE may be provided to the user through a number of visual, audible, tactile techniques, through indicators, summary graphs, coaching, and the like. In some embodiments, this guidance is integrated within a baseline set of voice instructions, tones, music, and the like which is altered based on a user BMIE matching, varying from within in a range of variance, cohering within a range of coherence, varying from a preferred BMIE. In some embodiments, guidance to adjust the user's movement, breath, or combination thereof is based on providing an instructor example, a symbol, audio feedback, overlay, text feedback, tactile feedback, feedback through a connected device such as heating/cooling, change in lighting, vibration, and the like. In some embodiments, the user is provided with an opportunity to “redo” an activity based on varying from a preferred BMIE.

In the example provided in FIG. 10, forms of BMIE representation include a rating 1020 which shows a coherence for the duration of the activity, which may also be a factor in a general rating 1060 related to the user activity participation, wellness, skill, focus, or the like. In some embodiments coherence rating 1020 may be a factor in a user badge, milestone, membership, or award system. In some embodiments, the coherence score 1020 is adjusted based on the user's current coherence rather and in some embodiments the coherence score 1020 is cumulative for a duration of the activity. Adjustment guidance 1030 may provide an indication of the user's current activity. In some embodiments, adjustment guidance provides information about characteristics of breath and movement that the user may need to adjust to match a preferred BMIE. The indicator may display both current activity and guidance simultaneously by adding color, flashing, overlays, and other indicators to show a relationship/similarity/difference between the user's current behaviour and the behaviour associated with a preferred BMIE. For example, one plus sign may be colored green and the second flashing red to indicate to the user to increase the speed of their inhalation and/or movement to achieve the preferred BMIE. Breath pattern indicator 1040 similarly may indicate the user's current breathing pattern as well as the preferred breathing pattern. In one embodiment, the breath indicator is green if the user is inhaling and matching a preferred inhalation, orange if the user is inhaling and partially matching a preferred inhalation, and red if the user is inhaling when the preferred breath pattern is an exhalation. In some embodiments, the visual indications may be modified based on user preferences.

Referring to FIG. 11 an example embodiment for breath-move interrelation evaluation and providing BMIE representations shows a smart mirror user device 10 with inputs such as camera and/or microphone 15A, and physiological sensor 15B where in addition to the screen display of user device 10 vibrating smart weights 17A providing and connected audio system 17B may provide additional means and techniques for providing the user with breath-move interrelation evaluation representations. For example, in one embodiment the vibrating smart weights 17A provide feedback when the user degree of coherence with a preferred BMIE is below a certain threshold and/or the audio system 17B provides an increased tempo in the soundtrack when the users motion is slower than the motion required for a preferred BMIE.

In this example, in addition to the user specific BMIE representations and information provided in lower panel 1140 instructor video 1010 is augmented with a BMIE representation 1120. In some embodiments a BMIE representation associated with another participant 1130 may also be available in the example shown with indicator 1135 such that the user may select the BMIE and/or other video associated with that individual. In some embodiments, the other participant may be associated with a specific skill level or training technique. The other participant, educator participant, model participant, and/or instructor may be based on a human engaged in the activity represented with streaming content, recorded content, or a combination or may be based on a generated avatar based on machine learning, a combination, or the like.

In this example, lower display portion 1140 provides user specific guidance for achieving a preferred BMIE 1145 which supplements general exercise guidance 1110 for performing the exercise. In addition to guidance, the user is provided with a real-time or near real-time visual indicator representing her current BMIE 1150, an at a glance star representation that shows in this example a rating of her correspondence to the preferred breath-move interrelation 1160, and additional key physiological data 1170 such as heartrate, heartrate variance, and the like. In some embodiments, representations may flash, change color, alter an overlay, add or remove visual elements, provide audio feedback, provide other tactile feedback, combinations or the like, in response to changes in the user BMIE pattern and/or variance of the user BMIE pattern from one or more preferred BMIE pattern. In some embodiments, the user may engage with the indicators to access additional BMIE or activity summary data, coaching, or other options. In some embodiments, the user is offered different activities, coaching, training, instructors, offers, purchase recommendations, provided in the future suggestions for activities, coaching, training, instructors, offers, purchase recommendations, based on their BMIE as a factor in determining these recommendations.

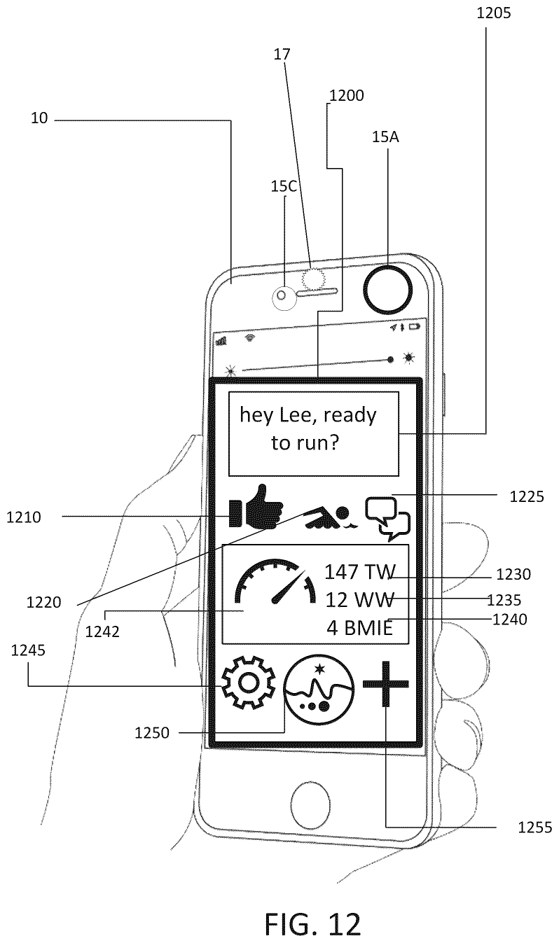

Turning to FIG. 12, an alternative example interface related to providing and generating breath-move interrelation evaluations and breath-move interrelation evaluation representations is shown. As shown in this figure, user device 10 with input camera 15A and input microphone 15C and output audio 17 in addition to display screen, may display application 1200 includes a breath-move interrelation evaluation representations 1250, ratings which may include BMIE values such as for example Total Wellness 1230, Weekly Wellness 1235, and a current BMIE rating 1240 where these values may be shown portrayed in combination in graphic forms such as gauge 1242.

In some embodiments, the user is provided with personalization 1205 which may be a component in the user selecting the activity or activity type associated with a BMIE, initiating a sensor associated with a BMIE, initiating an activity associated with a BMIE, and/or setting a preference. In some embodiments, the application 1200 includes determining a probable user activity based on user history, location, user sensor input, and/or a combination. Lee can click 1210 to select the proposed activity or click the swim button 1220 to specify an alternate activity. In some embodiments, the user may have access to coaching, training, chat, or other options by selecting to engage 1225. In some embodiments, the user is able to set preferences, calibrate sensors, and or set up a schedule for automatically generating BMIE 1245. In some embodiments, the same application that enables a user to select an activity may display a current BMIE 1250. In some embodiments, the user can access integrated or separately provided additional functionality such as a log of previous BMIE representations, a wellness recommendation system, membership system, retail platform, social media platform, wellness or fitness platform, wellness or fitness class and the like 1255.

In some embodiments, when the user is engaged in cyclical movement patterns (for example, walking, running, swimming, weight lifting, cycling, etc.) the BMIE calculation leverages these repeated movements (strides, strokes, reps, etc.) within the BMIE evaluation, representation, and or representation type selection. The generated BMIE can help the user align its movements with the cyclical movement patterns. For example, the output instructions for the BMIE can include guidance related to cyclical movement to help align strides or strokes of a user with cyclical movement patterns.

In certain activity contexts, specific breathing patterns have for some individuals been associated with one or more of a HR reduction, a perceived HR reduction, a reduction of stress and/or anxiety, a perceived reduction of stress and/or anxiety, improved cardio performance, and/or a perceived improved cardio performance. In some embodiments, activity model 65, BMIE model 60, and/or context model 75 comprise logic and patterns associated with one or more of increasing HR, decreasing HR, increasing HRV, decreasing HRV, distributing joint impact during movement patterns, improving endurance and/or stamina when engaged in a specific activity, activity intensity, or the like. In some embodiments, this includes pattern correspondence with one or more pattern such as 3 repeated movement (for example step) on inhale, 2 repeated movement (for example step) exhale; 2 repeated movement (for example step) on inhale, 1 repeated movement (for example step) exhale, 2 short inhalations and one long exhalation. In some embodiments, the logic and pattern selected may be based on an input related to HR, HRV, exertion, or the like.

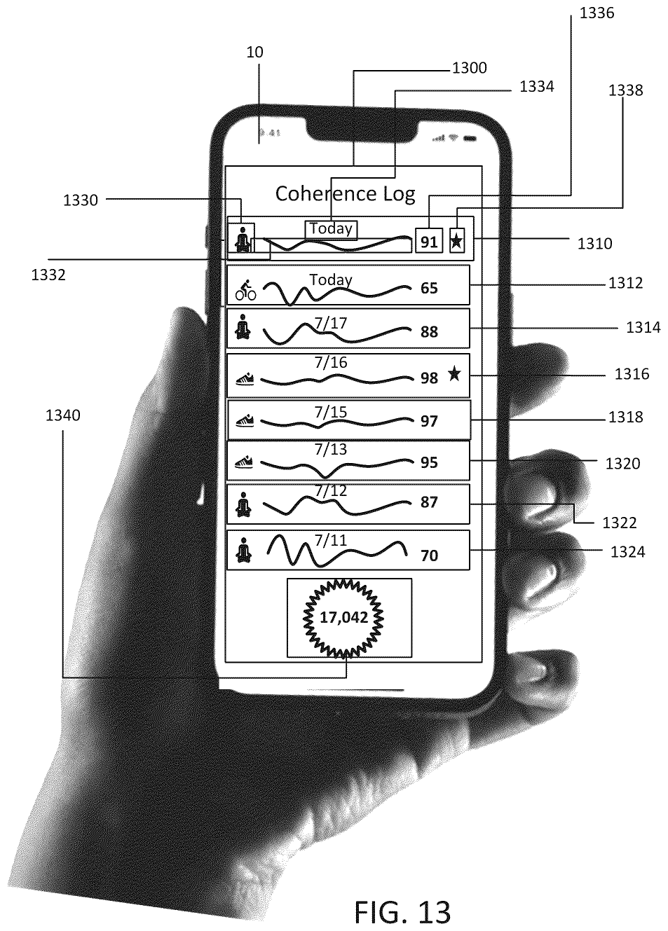

In some embodiments, the BMIE representation or BMIE representation type may be selected based on a physiological input, a physiological input in association with an activity, a physiological input in association with a breath characteristic, a physiological input in association with a movement characteristic, a physiological input in association with a BMIE. For example, some BMIE representations may require specific breath-move interrelation evaluation values and factors such as additional biometric data, a specific type of activity/movement associated with the BMIE, a specific activity intention associated with the activity and the like in order to be applicable, available and/or preferred. In some embodiments, one or more of these types of BMIE representation model are generated based on models associated with increasing HR, decreasing HR, increasing HRV, decreasing HRV. FIG. 13 provides an example user interface associated with generating and providing breath-move interrelation evaluations and breath-move interrelation evaluation representations. As shown in this figure, user device 10 displays application 1300 which includes a coherence log component. In some embodiments, application 1300 with coherence log component is integrated or interconnected with one or more of a fitness tracker application, a wellness recommendation system, membership system, retail platform, social media platform, wellness or fitness platform, wellness or fitness class and the like.

Example application 1300 which includes a coherence log component displays BMIE representations and other data associated with user current or past activity. Each entry 1310, 1312, 1314, 1316, 1318, 1320, 1322, 1324 is associated with an activity duration. The user interface includes elements such as activity type indicator 1330, activity date/time information 1334, activity BMIE coherence score 1336, BMIE graph representation 1332, and/or optional badges, awards, and/or milestones 1338, as well as a composite BM IE coherence rating for one or more activities 1340.

In some embodiments, activity type indicator 1330 identifies an activity type category (for example running or swimming), in some embodiments it identifies a specific activity within a category (for example sprinting or front crawl), and in some embodiments it identifies a specific training pattern or technique (for example fartlek or bilateral breathing), in some embodiments it identifies subcomponents such as stride or stroke, and in some embodiments the user is able to zoom in and/or zoom out to see BMIE representations associated with a selected level of activity specificity.

In some embodiments, for some activities for which monitoring BMIE values while performing the activity may not be preferable/appropriate, such as swimming, sprinting, skiing, and the like, the BMIE may be provided as a summary post-activity rather than during the activity. In some embodiments, for some activities the BMIE representation may be available real-time or near real-time as well as through a post-activity summary. In some embodiments, a different representation type is provided for a real-time or near real-time BMIE representation and a summary BMIE representation. In some embodiments, comparisons are made between more than one user BMIEs and provided as a representation.

These breath-move interrelation evaluation representations may be provided embedded within another application such as a fitness class application, an online retail application, a social media application, a membership tool application, a virtual environment, an augmented reality environment, a game environment, a mixed reality environment.

As is evident in these examples, a range of data and metadata inputs, may be evaluated to determine a breath-move interrelation, an evaluation of the breath-move interrelation, a breath-move interrelation evaluation representation, whether to provide a breath-move interrelation evaluation representation and what type of breath-move interrelation evaluation representation to provide, and/or one or more breath-move interrelation evaluation representation to provide to a user.

In some embodiments, the system for breath-move evaluation evaluates data associated with a user engaged in an activity such as such as sleeping, performing office type working, shopping online, watching a recorded event or live performance, and/or gaming. In some embodiments, a stillness measure is evaluated in relationship to breathing patterns to provide a BMIE. In some embodiments, a characteristic of the BMIE representation is associated with an adjustment to reduce the stillness measure.

In some embodiments, when evaluating a user breath-move interrelation associated with a activity such as performing computer-based office type work, shopping online, watching a recorded event or live performance, and/or gaming a stillness measure duration is evaluated in conjunction with the breath characteristics. In some embodiments, a breath move interaction evaluation representation may generate instructions to provide one or more of a one or more of visual symbol, overlay over a video depicting the user move, audio feedback, text, graph, summary, message, notification, rating value, rating value within a composite rating, lighting feedback, tactile feedback, vibration feedback, change in music, heating/cooling feedback associated with guidance to alter the user activity, user posture, suggest the user take a break, or the like. In some embodiments, the BMIE representation may generate instructions to automatically adjust a output or output characteristic in the user environment such as changing lighting, changing temperature, altering the height of a desk, pausing a user device, pausing a game or recorded event, or the like.

In some embodiments, when evaluating breathing patterns and movement associated with a user engaged in a sleep, a specific phase of sleep, a specific stage of sleep, pre-sleep, post-sleep, or sleep-proximate activity, the system evaluates breathing characteristics such as nose breathing, apnea, accelerated breathing, and movement characteristics such stillness, restless limbs, general restlessness, sleep walking, and the like. In some embodiments, the BMIE representation may generate instructions to automatically adjust an output or output characteristic in the user environment such as changing lighting, changing temperature, altering positioning of a bed, providing white noise, or the like. In some embodiments, a summary of BMIE over time associated with breathing patterns and movement for a user engaged in a sleep, pre-sleep, post-sleep, or sleep-proximate activity, the system evaluates is provided to the user after the activity within a message, reminder, tracker application, user interface within an application, or the like.

The word “a” or “an” when used in conjunction with the term “comprising” or “including” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

The terms “coupled”, “coupling” or “connected” as used herein can have several different meanings depending on the context in which these terms are used. For example, the terms coupled, coupling, or connected can have a mechanical or electrical connotation. For example, as used herein, the terms coupled, coupling, or connected can indicate that two elements or devices are directly connected to one another or connected to one another through one or more intermediate elements or devices via an electrical element, electrical signal or a mechanical element depending on the particular context. The term “and/or” herein when used in association with a list of items means any one or more of the items comprising that list.

As used herein, a reference to “about” or “approximately” a number or to being “substantially” equal to a number means being within +/−10% of that number.

The technical solution of embodiments may be in the form of a software product. The software product may be stored in a non-volatile or non-transitory storage medium. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

While the disclosure has been described in connection with specific embodiments, it is to be understood that the disclosure is not limited to these embodiments, and that alterations, modifications, and variations of these embodiments may be carried out by the skilled person without departing from the scope of the disclosure.

It is furthermore contemplated that any part of any aspect or embodiment discussed in this specification can be implemented or combined with any part of any other aspect or embodiment discussed in this specification.

1. A device for generating output instructions for a breath-move interrelation evaluation, the device comprising:
a processing system having one or more one hardware processors and one or more memories coupled with the one or more processors programmed with executable instructions to cause the device to:
transmit control signals to one or more sensors to perform measurements of a user associated with one or more activity of the user;
obtain input data from the measurements of the user and contextual metadata, wherein the input data comprises data characterizing a user breath pattern and data characterizing a user movement, wherein the contextual metadata identifies one or more of the user, the activity, an activity type, an activity class, an activity series, and an activity group;
compute a set of interrelations between the data characterizing the user breath pattern and the data characterizing the user movement;
generate the output instructions for a breath-move interrelation evaluation representation based on the set of interrelations, the breath-move interrelation evaluation being associated with the one or more activity of the user; and
transmit the output instructions to provide the breath-move interrelation evaluation representation at a user interface or store the breath-move interrelation evaluation representation in the one or more memories, the output instructions to activate or trigger the user interface to present the breath-move interrelation evaluation representation;
wherein the device communicates with the one or more sensors coupled to one or more transmitters, wherein the one or more sensors perform the measurements of the user associated with the one or more activity of the user, the one or more transmitters transmit the measurements to the device.

2. The device of claim 1 wherein the processing system generates a baseline breath-move interrelation evaluation associated with the user or the activity, wherein the processing system generates the output instructions for the breath-move interrelation evaluation representation by comparing the breath-move interrelation evaluation representation to the baseline breath-move interrelation evaluation to determine that the breath-move interrelation evaluation representation varies from the baseline breath-move interrelation evaluation within a threshold.

3. The device of claim 1 wherein the activity involves cyclical movement and wherein the output instructions for the breath-move interrelation evaluation comprise guidance related to the cyclical movement.

4. The device of claim 1 wherein the processing system evaluates the set of interrelations between the data characterizing the user breath pattern and the data characterizing the user movement against a preferred interrelation between the data characterizing the user breath pattern and the data characterizing the user movement, wherein the processing system identifies the preferred interrelation using the contextual metadata that identifies the one or more of the user, the activity, the activity type, the activity class, the activity series, and the activity group, wherein the processing system identifies the preferred interrelation using a preferred breath-move interrelation model or a model which comprises one or more preferred breath-move interrelation representation type.

5. The device of claim 1 wherein the activity is selected from the group consisting of sleep, exercise, a wellness activity, work, shopping, watching an event or performance, and gaming.

6. The device of claim 1 wherein the instructions to provide the breath-move interrelation evaluation representation at the user interface of the electronic device provides one or more selected from the group of a symbolic visual representing the breath-move interrelation representation as a visual component of the user interface, visual symbol, overlay over a video depicting the user movement, audio feedback, text, graph, summary, message, notification, rating value, rating value within a composite rating, lighting feedback, tactile feedback, vibration feedback, change in music, and heating/cooling feedback.

7. The device of claim 1 wherein the processing system computes the set of interrelations between the data characterizing the user breath pattern and the data characterizing the user movement using a machine learning model for interrelation between breathing patterns and movement.

8. The device of claim 1 wherein the processing system uses a machine learning model comprising one or more preferred breath-move interrelations types to evaluate the set interrelations between the data characterizing the user breath pattern and the data characterizing the user movement.

9. The device of claim 1 wherein the processing system extracts, from the data characterizing the user movement, one or more features selected from the group of an eccentric aspect, a concentric aspect, a stillness threshold aspect, a direction of movement, jerk, cadence, center of gravity, center of pressure, movement duration, movement phase, smoothness, movement associated with a specific body portion and/or limb, an anaerobic threshold aspect, an aerobic threshold, a stillness measure and computes the set of interrelations using the one or more extracted features.

10. The device of claim 1 wherein the processing system uses the data characterizing the user movement to identify an eccentric aspect and associate an inhalation logic, and to identify a concentric aspect and associate an exhalation logic.

11. The device of claim 1 wherein the processing system extracts, from the data characterizing the user breath pattern, one or more features selected from the group of mouth breathing, nose breathing, depth of inhalation, belly expansion, belly tension, consistency, oxygen levels, velocity, rate, volume, coherence, and computes the set of interrelations using the one or more extracted features.

12. The device of claim 1 wherein the processing system identifies one or more of another user, user group, user type, and compares the set of interrelations against a second interrelation associated with one or more of the other user, the user group, the user type, a previously generated interrelation for the user, an exemplary user, a generalized model based on a set of users.

13. The device of claim 1 wherein the processing system evaluates a value associated with a breath-move interrelation evaluation representation and changes content for the user interface with content based on the value by one or more of presenting, removing, unlocking, and customizing, and wherein the content is one or more of a personalization, a feature, a retail offer, a retail experience, a user profile, a user wish list of products or services, a class, a group activity, a workshop, a coaching session, a video, a song, a graphic user interface skin, a performance event, community event, an exercise class, an avatar, an avatar's clothing, an avatar accessory, a conversational interaction, a notification, a pop-up suggestion, an alarm, a badge, a group membership.

14. The device of claim 1 wherein the output instructions to provide the breath-move interrelation evaluation representation provide guidance to shift one or more of the user breath pattern, the user movement pattern to increase correspondence with a preferred breath-move interrelation evaluation.

15. The device of claim 1 wherein the breath-move interrelation evaluation representation is associated with one or more types of breath-move interrelation evaluation representations, wherein the processing system generates the output instructions for the breath-move interrelation evaluation representation by selecting a breath-move interrelation evaluation representation type based on one or more of a user location, a user device, a user group membership, a user device type, a user system type, a user preference, and the activity.

16. The device of claim 1 wherein the processing system is part of one or more selected from the group of an exercise apparatus, exercise platform, a smart mirror, smart phone, a computer, a tablet, a smart exercise device, a fitness tracker, a connected fitness system, a connected audio system, a connected lighting system, a smart exercise device, a component within a connected smart exercise system, a smart mat, a smart watch, a smart sensor, a virtual reality headset, an augmented reality headset, a haptic glove, a haptic garment, a game controller, a hologram projection system, an autostereoscopic projection system, mixed reality devices, virtual reality devices, an augmented reality device, a metaverse headset, a retail platform, a recommendation system, and a social networking community system, gaming platform system, membership system, activity tracking system, machine learning system, a virtual reality environment, an augmented reality environment, a mixed-reality environment, or a combination thereof.

17. The device of claim 1 wherein the processing system communicates with a messaging system to provide the breath-move interrelation evaluation representation through one or more of email, SMS message, MMS message, social media notification, notification message on the user interface.

18. The device of claim 1 wherein the one or more of the sensors is one or more of a camera, a video camera, a microphone type sensor, a heart rate monitor, a breathing monitor, a blood glucose monitor, a humidity sensor, an oximetry sensor, an electronic implant, an EEG, a brain-computer interface, an accelerometer, a restive sensor, a gyroscope, an inertial sensor, a Global Positioning System (GPS) sensor, a Passive Infrared (PIR) sensor, an active infrared sensor, a Microwave (MW) sensor, an area reflective sensor, a lidar sensor, an infrared a spectrometry sensor, an ultrasonic sensor, a vibration sensor, an echolocation sensor, a proximity sensor, a position sensor, an inclinometer sensor, an optical position sensor, a laser displacement sensor, a multimodal sensor, a pressure sensor, an acoustic sensor.

19. A non-transitory computer readable medium with instructions stored thereon, that when executed by a hardware processor causes the processor to: transmit control signals to one or more sensors to perform measurements of a user associated with one or more activity of the user; receiving input data characterizing a user breathing pattern from the measurements and input data characterizing a user movement from the measurements, calculating a set of interrelations between the input data characterizing the user breath pattern and the input data characterizing the user movement; generating output instructions for a breath-move interrelation evaluation representation based on the set of interrelations; and transmitting the output instructions to provide the breath-move interrelation evaluation representation at a user interface of an electronic device or storing an indication of the breath-move interrelation evaluation representation in memory.

20. A computer implemented method for generating output instructions for a breath-move interrelation evaluation, the method comprising:
transmitting control signals to one or more sensors to perform measurements of a user associated with one or more activity of the user and synchronize the one or more one or more sensors performing measurements;
receiving, using at least one hardware processor and the one or more sensors to perform measurements, input data that comprises data characterizing a user breath pattern;
receiving, using the at least one hardware processor and the one or more sensors to perform measurements, input data that comprises data characterizing a user movement;
receiving, using the at least one hardware processor, metadata related to the data characterizing the user breath pattern and the data characterizing the user movement;
computing using the at least one hardware processor, a set of interrelations based on the data characterizing the user breath pattern and the data characterizing the user movement;
generating, based on the calculated interrelations, the output instructions to provide the breath-move interrelation evaluation; and
transmitting the output instructions to provide the breath-move interrelation evaluation representation at a user interface of an electronic device or store the breath-move interrelation evaluation representation in memory.
